(19) United States
(12) Patent Application Publication
(10) Pub. No.: US 2004/0071703 A1
(43) Pub. Date: Apr. 15, 2004

(54) Multicomponent mscramm vaccine

(76) Inventors: Eric L. Brown, Houston, TX (US); Magnus Hook, Houston, TX (US); William S. Probert, Novato, CA (US); Jung-Hwa Kim, Houston, TX (US)

Correspondence Address: Raymond Reese, Howrey Simon Arnold & White, 750 Bering Drive, Houton, TX, 77057-2198, US

(21) Appl. No.: 10/380,191

(22) PCT Filed: Sep. 10, 2001
(86) PCT No.: PCT/US01/28541

(51) Int. Cl. A61K 39/395; C07K 16/18

(52) U.S. Cl. 424/145.1; 530/388.25

(57) ABSTRACT

Borrelia burgdorferi, the causative agent of Lyme disease (LD), expresses two decorin-binding adhesins (DbpA and DbpB) and at least one fibronectin-binding adhesin (BBK32). Mice deficient in decorin have been shown to have an increased resistance to Borrelia infection in both tick- and needle-inoculation models compared to control mice. BBK32 was used to vaccinate both decorin-deficient and wild-type mice prior to B. burdorferi infection. Decorin-deficient, BBK32-immunized mice had fewer positive ear, bladder and joint cultures compared to genotype and wild-type controls. These data suggest that a vaccine composed of BBK32/DbpA or components thereof may emerge as an effective vaccine treatment against LD.

This application claims priority of Provisional Application No. 60/231,133, filed Sep. 8, 2000, the content of which is incorporated by reference.

### BACKGROUND OF THE INVENTION

1. Description of the related art

Lyme disease (Steere, 1989), or Lyme borreliosis, is transmitted by ticks, particularly of the genus Ixodes, and caused by spirochetes of the genus Borrelia. Lyme disease agents, that is Borreliae isolated from humans or animals with clinical Lyme disease, are currently classified into at least two phylogenetic groups: B. burgdorferi sensu stricto, strain B31, and B. burgdorferi sensu strictu and lato. The genotypic and phenotypic variation among Lyme disease agents supporting the designation of these phylogenetic sub-groupings is a major complicating factor for the design of effective vaccines or immunotherapeutic strategies for Lyme disease.

Lyme disease is transmitted through the bite of a tick, which attaches itself to the host and, upon feeding, deposits the spirochetes into the dermis of the skin. In the skin, B. burgdorferi replicates before endovascular dissemination to organs. Typically, an annular spreading skin lesion, erythema migrans, forms at the site of the tick bite. Early symptoms of Lyme disease are flu-like and may include fatigue and lethargy. Left untreated, Lyme disease can develop into a chronic, multisystemic disorder involving the skin, joints, heart, and central nervous system.

Once deposited in the dermis, the spirochetes become associated with and appear to colonize the collagen fibers. Skin is the most consistent site of spirochete-positive culture. In persistent infection, the skin may provide a protective niche for replication, thereby acting as a reservoir of spirochetes for subsequent distribution to other tissues.

As B. burgdorferi disseminates to other organs, the organisms appear to localize to the extracellular spaces of these tissues as well. Bacterial adherence to host tissue is a critical first step in the initiation of most bacterial infections. The interactions between the host's extracellular matrix proteins (ECM) and invading bacteria are carried out by adhesins termed MSCRAMMs (microbial surface components recognizing adhesive matrix molecules).

In several organs, including tendon (Barthold et al., 1993; 1991), ligament (Haupl et al., 1993), heart (Zimmer et al., 1990), and muscle (Barthold et al., 1992; Duray, 1992), B. burgdorferi spirochetes are found primarily in close association with collagen fibers, suggesting that this association is an important mechanism of tissue adherence in different stages of infection. It has recently been determined that B. burgdorferi spirochetes bind specifically to a protein associated to collagen, Decorin (Dcn). Lyme disease is typically treated with antibiotics, which are generally effective in the early stages of the disease. Later stages involving cardiac, arthritic, and nervous system disorders are often non-responsive.

2. Existing Vaccines for Prevention of Lyme Disease

Several proteins present on the outer surface of B. burgdorferi have been identified, including OspA (31 kDa), OspB (34 kDa), OspC (22 kDa), OspD, OspE, and OspF. Laboratory studies have shown that passively-administered antibodies (Schaible et al., 1990) reactive with the B. burgdorferi outer surface protein A (OspA), or immunization with recombinant OspA (Fikrig et al., 1990), protect mice from challenge with in vitro-grown or tick-borne B. burgdorferi. Based largely on the protective efficacy of experimental OspA vaccines in rodent models of Lyme borreliosis, three monovalent OspA-based vaccines are currently in use. However, recent findings suggest that broad, sustained protection of humans may be difficult to achieve with vaccines based solely on OspA.

Four observations, however, suggest that OspA-based vaccines may prove to have limited efficacy in treating Lyme disease in humans:

a) Modulation of OspA expression by B. burgdorferi may limit the site of action of OspA-specific antibodies to spirochetes residing in the tick midgut as these antibodies are ineffective shortly after infection;

b) Human immune responses to OspA subunit vaccines have not matched those of rodents in level or duration; and

c) OspA is serologically diverse, particularly among European and Asian B. andersonii and B. azfelii isolates. Reactivity with panels of OspA monoclonal antibodies (mAbs), and DNA sequence analysis has shown that as many as seven different OspA subgroups can be distinguished (Wilske et al., 1991; 1993).

d) Cross-reactive epitopes between OspA and the lymphocyte function antigen-1 (LFA-1) have been observed and are one explanation for patients classified as treatment resistant.

Moreover, these variations will no doubt affect the cross-protection to be anticipated with OspA vaccines. Cross-protection was seen by one group using an immunocompetent mouse model (Fikrig et al., 1995), but cross-protection was weak or absent in SCID mouse or hamster models used by other (Schaible et al., 1993; Lovrich et al., 1995). An additional concern is that as many as 10% of B. burgdorferi isolates fail to express OspA in culture (Wilske et al., 1991; to 1993).

Another problem with the use of OspA as antigens for stimulation of an immune response in an affected patient is the fact that OspA protein is either poorly immunogenic in humans, or not expressed by B. burgdorferi in vivo until late in infection. Lyme disease patients, mice, hamsters, and dogs infected by tick bite or low-doses of cultured B. burgdorferi fail to mount substantial anti-OspA immune responses for many months following infection although they do mount early responses to other B. burgdorferi antigens (flagellin, OspC, etc.) (Steere, 1989; Barthold and Bockenstedt, 1993). OspA is expressed by B. burgdorferi within ticks (Barbour et al., 1983), but detection of OspA on Borreliae in tissue early after infection is difficult. Passive immunization of mice with OspA antibody (Schaible et al., 1990), or immunization with recombinant OspA, after challenge does not eliminate infection and only partially alters disease. Unfortunately, OspA-immunized mice are not protected from a challenge with host-adapted spirochetes delivered in the form of skin biopsy transplants from infected mice (Barthold et al., 1995). This would be explained by down-regulation of OspA expression by Borreliae shortly after initiation of feeding by the tick.

de Silva et al. (1996) demonstrated that when OspA-specific antibodies were administered to mice before or at the time of attachment of Borrelia-infected ticks these mice were protected from spirochetal infection. However, when OspA-specific antibody was administered 48-hr after tick attachment no protection was observed.

Modulation of Borrelia antigen expression within feeding ticks has recently been reported for OspC; initially low in resting ticks, OspC levels increase on B. burgdorferi after initiation of tick feeding (Schwan et al., 1995). OspC might appear to be a promising in vivo target, but its high level of antigenic variation complicates its development as a vaccine (Probert and LeFebvre, 1995).

In vitro cultivation of B. burgdorferi suggests that the genes for OspA and OspC are inversely regulated. Preliminary findings of some researchers do suggest that OspA levels similarly decrease after initiation of tick feeding. The current Lyme vaccine (LYMErix) is a formulation based on the outer surface protein A (OspA) of B. burgdorferi. OspA is expressed within the arthropod vector and is quickly down-regulated following transmission from the tick to the mammalian host. This requires OspA-derived vaccines to elicit a significant antibody response against OspA so that OspA-reactive antibodies serve to prevent transmission from the tick. Low antibody titers against OspA will not prevent spirochete transmission. Current OspA-derived vaccines also require a minimum of 2 boosts over a 1-year period before maximum efficacy can be reached. Lastly, cross-reactive epitopes between OspA and the lymphocyte function antigen-1 (LFA-1) are one explanation for patients classified as treatment resistant. These individuals may, in fact, be suffering from Borrelia/OspA-induced autoimmunity. In addition, high anti-OspA antibody titers have been linked with arthritis formation in Lyme patients.

Telford et al. (1995) describes the efficacy of human Lyme disease vaccine formulations in a mouse model. The authors speculate that “it is likely that the titer of circulating antibodies to OspA critically determines protection because of the unique mode of action of antispirochetal immunity, wherein antibody or other effectors interfere with the process of transmission within the gut of the infecting tick, before inoculation of the pathogen.” Consistent with this hypothesis it has been shown that anti-Borrelia serum can protect mice from infection by tick bite if administered within two days after initiation of feeding by Borrelia-infected ticks, but not when passively administered at later times (Shih et al., 1995). The antibody levels in response to recombinant OspA subunit vaccinations seen to date in Phase II trials have been moderate, with serum ELISA titers <3,000, and drop off to near baseline levels within five months (Keller et al., 1994). The results in these studies indicate that it will be necessary to include additional antigens to achieve a protective vaccine for Lyme disease. 4. Deficiencies in the Prior Art

It is clear that while several approaches to the treatment of bacterial diseases have experienced some success, many problems remain, including antibiotic resistance, variability of antigens between species and species variation through mutation of antigens, as well as the need to protect susceptible groups such as young children, the elderly and other immunocompromised patients. Thus, there exists an immediate need for an effective treatment for B. burgdorferi, and vaccines against the causative agent of Lyme disease.

Although attempts have been made to utilize the Osps as vaccines to confer protection against B. burgdorferi, the results have been disappointing. Because these proteins have demonstrated stain specificity, e.g., variance among isolates and among different passages, and some lack of cross protection between strains, their potential use as vaccines remains very limited.

Because currently known antigens are not sufficient to elicit a protective immune response over a broad spectrum of B. burgdorferi strains, there continues to be an urgent need to develop novel prevention and treatment methods as well as novel antigens able to elicit a broad-spectrum immune response and useful diagnostic methods for the prevention, treatment, and diagnosis of Lyme disease.

### SUMMARY OF THE INVENTION

The present invention overcomes one or more of these and other drawbacks inherent in the prior art by providing novel compositions and methods for their use in the treatment of Lyme disease using non-antibiotic strategies. Disclosed are methods for the use of novel peptides and antibody compositions in the treatment of Lyme disease mediated by the inhibition of B. burgdorferi binding to the host cell ECM components, fibronectin and decorin. Also disclosed are methods for active and passive immunization against B. burgdorferi using a combination of two Borrelia adhesins; a fibronectin binding protein (FBP) (BBK32) and the decorin binding protein (DBP) type A (DbpA). Particular aspects of the invention relate to novel peptides and epitopes, and methods for the use of such peptides in therapeutic regimens.

Biochemical and immunological characterization of the B. burgdorferi DBPs and FBPs show that Lyme disease vaccines comprising DbpA and/or the newly characterized BBK32 compositions overcome the limitations of the prior art involving OspA and, are, indeed, superior to those formulations based on OspA regimens.

Specifically, this invention encompasses various compositions comprising a decorin binding protein or decorin binding peptide and a fibronectin binding protein or fibronectin binding peptide. Most preferred is the composition wherein said decorin binding protein or decorin binding peptide comprises the amino acid sequence from SEQ ID NO:4 and wherein said fibronectin binding protein or fibronectin binding peptide comprises the amino acid sequence from SEQ ID NO:2.

Further envisioned in this application is a method to elicit an immunological response in an animal by injecting said animal with a composition comprising a decorin binding protein or decorin binding peptide and a fibronectin binding protein or fibronectin binding peptide. Most preferred is the method wherein said decorin binding protein or decorin binding peptide comprises the amino acid sequence from SEQ ID NO:4 and wherein said fibronectin binding protein or fibronectin binding peptide comprises the amino acid sequence from SEQ ID NO:2.

Further envisioned in this application is a method for preventing Lyme disease by injecting an animal an effective amount of a composition comprising a decorin binding protein or decorin binding peptide and a fibronectin binding protein or fibronectin binding peptide. Most preferred is the method wherein said decorin binding protein or decorin binding peptide comprises the amino acid sequence from SEQ ID NO:4 and wherein said fibronectin binding protein or fibronectin binding peptide comprises the amino acid sequence from SEQ ID NO.2.

### BRIEF DESCRIPTION OF THE DRAWINGS

The drawings form part of the present specification and are included to further demonstrate certain aspects of the present invention. The invention may be better understood by reference to one or more of these drawings in combination with the detailed description of specific embodiments presented herein.

FIG. 1A: Effect of BBK32—or control-vaccination on wild type mice.

Wild-type BALB/c mice (n=5 mice/group) were infected i.d. with 10⁴ B. burgdorferi after vaccination with BBK32 or control proteins. Two weeks after infection, the mice were sacrificed and ear, bladder, and one joint were inoculated into BSK II medium. After two weeks in culture, cultures were examined for the presence of spirochetes.

FIG. 1B: Effect of BBK32—or control-vaccination on Decorin-deficient mice

Decorin-deficient BALB/c mice (n=5 mice/group) were infected i.d. with 10⁴ B. burgdorferi after vaccination with BBK32 or control proteins. Two weeks after infection, the mice were sacrificed and ear, bladder, and one joint were inoculated into BSK II medium. After two weeks in culture, cultures were examined for the presence of spirochetes.

FIG. 2A: Blood cultures after BBK32—or BBK32/DbpA-vaccination on wild type mice.

Wild-type mice (n=5 mice/group) were vaccinated with BBK32 or with BBK32/DbpA and then infected with 10⁴ B. burgdorferi. Blood was collected one week post infection and cultured for two to three weeks for the presence of Borrelia.

FIG. 2B: Tissue cultures after BBK32—or BBK32/DbpA-vaccination on wild type mice.

Wild-type mice (n=5 mice/group) were vaccinated with BBK32 or with BBK32/DbpA and then infected with 10⁴ B. burgdorferi. One week post infection, the mice were sacrificed and joints, heart, ear, and bladder samples were aseptically removed and cultured for the presence of Borrelia (day 14 post infection).

FIG. 3A: Antibody response to DbpA 4 weeks post primary immunization.

Wild-type mice (n=5 mice/group) were vaccinated with DbpA and the antibody response was measured. Each data point represents the mean absorbance from triplicate wells from individual mice at 405 nm minus the substrate control.

FIG. 3B: Antibody response to BBK32 4 weeks post primary immunization.

Wild-type mice (n=5 mice/group) were vaccinated with BBK32 and the antibody response was measured. Each data point represents the mean absorbance from triplicate wells from individual mice at 405 nm minus the substrate control.

FIG. 4: Antibody response to DbpA 1 week post secondary immunization.

Wild-type mice (n=5 mice/group) were vaccinated with DbpA and the antibody response was measured. Each data point represents the mean absorbance from triplicate wells from individual mice at 405 nm minus the substrate control.

FIG. 4B: Antibody response to BBK32 1 week post secondary immunization.

Wild-type mice (n=5 mice/group) were vaccinated with BBK32 and the antibody response was measured. Each data point represents the mean absorbance from triplicate wells from individual mice at 405 nm minus the substrate control.

### DETAILED DESCRIPTION OF THE INVENTION

1. Nucleic Acid Compositions

The invention provides nucleic acid sequences encoding DbpA and BBK32 proteins. As used herein, a “dbp gene” means a nucleic acid sequence encoding a DBP and a “fbp gene” means a nucleic acid sequence encoding a FBP protein. Preferred dbp genes include the dbpA gene, in particular those from B. burgdorferi sensu strictu and lato. Preferred fbp genes include the fbp gene, in particular those from B. burgdorferi sensu strictu and lato.

A preferred nucleic acid sequence encoding a dbpA gene is the nucleotide sequence of SEQ ID NO:3 of the B. burgdorferi dbpA gene, or strain variants or active fragments thereof. A preferred nucleic acid sequence encoding a fbp gene is the nucleotide sequence of SEQ ID NO:1 of the B. burgdorferi bbk32 gene, or strain variants or active fragments thereof.

It is expected that the genes encoding DBPs and FBPs will vary in nucleic acid sequence from strain to strain, but that the variation in nucleic acid sequence will not preclude hybridization between sequences encoding the DBPs and FBPs of each strain under moderate to strict hybridization conditions. It is also contemplated that the genes encoding DbpAs and BBK32 from various strains may vary in nucleic acid sequences, but that the variation will not preclude hybridization between sequences encoding a DbpA or BBK32 from each strain under moderate to strict hybridization conditions.

As used herein, a strain variant of a DBP or FBP means any polypeptide encoded, in whole or in part, by a nucleic acid sequence which hybridizes under moderate to strict hybridization conditions to the nucleic acid sequence of SEQ ID NO:3 or SEQ ID NO:1.

Likewise, as also used herein, a strain variant of a DBP or FBP means any polypeptide encoded, in whole or in part, by a nucleic acid sequence which hybridizes under moderate to strict hybridization conditions to the nucleic acid sequence of SEQ ID NO:3 or SEQ ID NO:1.

One of skill in the art will understand that strain variants of DBPs or FBPs include those proteins encoded by nucleic acid sequences which may be amplified using one or more of the dbpA-encoding nucleic acid sequences of SEQ ID NO:3 or one or more of the bbk32-encoding nucleic acid sequence of SEQ ID NO:1.

In related embodiments, the invention also comprises strain variants of DBPs and nucleic acid segments encoding DBPs, in particular, the dbpA genes which encode the DbpA protein. Strain variants are those nucleic acid compositions and polypeptide compositions expressed by various strains of B. burgdorferi and related Borrelias including B. burgdorferi sensu strictu and lato, which specifically encode DBPs.

In related embodiments, the invention also comprises strain variants of FBPs and nucleic acid segments encoding FBPs, in particular, the bbk32 genes which encode the BBK32 protein. Strain variants are those nucleic acid compositions and polypeptide compositions expressed by various strains of B. burgdorferi and related Borrelias including B. burgdorferi sensu strictu and lato, which specifically encode FBPs.

As used herein, a DBP means a purified and isolated protein including a strain variant or an active fragment thereof, derived from B. burgdorferi or a similar species which induces Lyme disease in a similar way, and having the ability to bind Decorin (Dcn). Preferably, a DBP is a DbpA protein encoded by a nucleic acid sequence contained within the B. burgdorferi DNA shown in SEQ ID NO.3.

As used herein, a FBP means a purified and isolated protein including a strain variant or an active fragment thereof, derived from B. burgdorferi or a similar species which induces Lyme disease in a similar way, and having the ability to bind Fibronectin (Fbn). Preferably, a FBP is a BBK32 protein encoded by a nucleic acid sequence contained within the B. burgdorferi DNA shown in SEQ ID NO:1.

In the present invention, a composition comprising a DBP is also understood to comprise one or more polypeptides that are immunologically reactive with antibodies generated against B. burgdorferi sensu strictu and lato, or related Borreliae. and in particular antibodies generated against a DbpA protein, particularly those encoded by the dbpA nucleic acid sequences of SEQ ID NO:3 or to active fragments, or to strain variants thereof.

In the present invention, a composition comprising a FBP is also understood to comprise one or more polypeptides that are immunologically reactive with antibodies generated against B. burgdorferi, sensu strictu and lato, or related Borreliae and in particular antibodies generated against a BBK32 protein, particularly those encoded by the bbk32 nucleic acid sequences of SEQ ID NO:1 or to active fragments, or to strain variants thereof.

Likewise, a composition comprising a DBP of the present invention is understood to comprise one or more polypeptides that are capable of eliciting antibodies that are immunologically reactive with one or more DbpA proteins encoded by one or more dbpA nucleic acid sequences contained in SEQ ID NO:3 or to active fragments, or to strain variants thereof, or to one or more nucleic acid sequences which hybridize to one or more of these sequences under conditions of moderate to high stringency.

Likewise, a composition comprising a FBP of the present invention is understood to comprise one or more polypeptides that are capable of eliciting antibodies that are immunologically reactive with one or more BBK32 proteins encoded by one or more bbk32 nucleic acid sequences contained in SEQ ID NO:1 or to active fragments, or to strain variants thereof, or to one or more nucleic acid sequences which hybridize to one or more of these sequences under conditions of moderate to high stringency.

A composition comprising a DBP of the present invention is also understood to comprise one or more polypeptides that elicit an immune response in a treated animal, this immune response being effective to lessen or prevent symptomatic disorders associated with Lyme disease or related borreliosis, or which polypeptides are capable of eliciting antibodies that are immunologically reactive with a DbpA protein encoded by a nucleic acid sequence of SEQ ID NO:3, or with an active fragment, or with one or more strain variants thereof.

A composition comprising a FBP of the present invention is also understood to comprise one or more polypeptides that elicit an immune response in a treated animal, this immune response being effective to lessen or prevent symptomatic disorders associated with Lyme disease or related borreliosis, or which polypeptides are capable of eliciting antibodies that are immunologically reactive with a BBK32 protein encoded by a nucleic acid sequence of SEQ ID NO:1, or with an active fragment, or with one or more strain variants thereof.

As used herein, a decorin binding peptide of a DBP includes a whole or a portion of a DBP which is modified by conventional techniques, e.g., mutagenesis, or by addition, deletion, or substitution, but which active fragment exhibits substantially the same structure and function as a native DBP as described herein. For example, portions of the protein not required to block adherence of B. burgdorferi to Dcn may be deleted or altered; additions to the protein may be made to enhance the protein's antigenicity according to conventional methods.

As used herein, a fibronectin binding peptide of a FBP includes a whole or a portion of a FBP which is modified by conventional techniques, e.g., mutagenesis, or by addition, deletion, or substitution, but which active fragment exhibits substantially the same structure and function as a native FBP as described herein. For example, portions of the protein not required to block adherence of B. burgdorferi to Fbn may be deleted or altered; additions to the protein may be made to enhance the protein's antigenicity according to conventional methods.

As used herein, a method for preventing Lyme disease means administering a composition comprising a DBP which prevents or lessens adhesion of B. burgdorferi to Dcn, or prevents or lessens adhesion the severity of any of the disorders associated with B. burgdorferi infection, including erythema migrans, arthritis, carditis, neurological disorders, or any other Lyme disease-related disorder.

As used herein, a method for preventing Lyme disease means administering a composition comprising a FBP prevents or lessens adhesion of B. burgdorferi to Fbn, or prevents or lessens adhesion the severity of any of the disorders associated with B. burgdorferi infection, including erythema migrans, artritis, carditis, neurological disorders, or any other Lyme disease-related disorder.

The term “biologically functional equivalent” is well understood in the art. Accordingly, sequences that have between about 70% and about 80%; or more preferably, between about 81% and about 90%; or even more preferably, between about 91% and about 99%; of amino acids that are identical or functionally equivalent to the amino acids disclosed herein, will be sequences that are “essentially as set forth in SEQ ID NO:4 or SEQ ID NO:2”.

It will also be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various upstream or downstream regulatory or structural genes. If desired, one may also prepare fusion proteins and peptides, e.g., where the DBP or FBP coding regions are aligned within the same expression unit with other proteins or peptides having desired functions, such as for purification or immunodetection purposes (e.g., proteins that may be purified by affinity chromatography and enzyme label coding regions, respectively).

Recombinant vectors form further aspects of the present invention. Particularly useful vectors are contemplated to be those vectors in which the coding portion of the DNA segment, whether encoding a full length protein or smaller peptide, is positioned under the control of a promoter. The promoter may be in the form of the promoter that is naturally associated with a DBP gene, as may be obtained by isolating the 5′ non-coding sequences located upstream of the coding segment, for example, using recombinant cloning and/or PCR.TM. technology, in connection with the compositions disclosed herein.

In other embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with a dbp gene in its natural environment. Such promoters may include dbpA or bbk32 promoters normally associated with other genes, and/or promoters isolated from any bacterial, viral, eukaryotic, or mammalian cell. Naturally, it will be important to employ a promoter that effectively directs the expression of the DNA segment in the cell type, organism, or even animal, chosen for expression. The use of promoter and cell type combinations for protein expression is generally known to those of skill in the art of molecular biology, for example, see Sambrook et al. (1989). The promoters employed may be constitutive, or inducible, and can be used under the appropriate conditions to direct high level expression of the introduced DNA segment, such as is advantageous in the large-scale production of recombinant proteins or peptides.

Prokaryotic expression of nucleic acid segments of the present invention may be is performed using methods known to those of skill in the art, and will likely comprise expression vectors and promotor sequences such as those provided by tac, trp, lac, lacUV5 or T7. When expression of the recombinant DBP or FBP proteins is desired in eukaryotic cells, a number of expression systems are available and known to those of skill in the art. An exemplary eukaryotic promoter system contemplated for use in high-level expression is the Pichia expression vector system (Pharmacia LKB Biotechnology).

In connection with expression embodiments to prepare one or more recombinant FBPs, DBPs, DbpA- or BBK32-derived peptides, it is contemplated that longer DNA segments will most often be used, with DNA segments encoding an entire DBP or FBP or one or more functional domains, epitopes, ligand binding domains, subunits, etc. therefore being most preferred. However, it will be appreciated that the use of shorter DNA segments to direct the expression of a DBP, a DBP-derived peptide or epitopic core region, a FBP or a FBP-derived peptide or epitopic core region, such as may be used to generate anti-DbpA or anti-BBK32 antibodies, also falls within the scope of the invention. DNA segments that encode peptide antigens from about 10 to about 100 amino acids in length, or more preferably, from about 10 to about 50 amino acids in length are contemplated to be particularly useful.

The dbpA and bbk32 genes and DNA segments derived therefrom may also be used in connection with somatic expression in an animal or in the creation of a transgenic animal. Again, in such embodiments, the use of a recombinant vector that directs the expression of the full length (or active) DBP or FBP is particularly contemplated. Expression of dbpA and or bbk32 transgene in animals is particularly contemplated to be useful in the production of anti-DbpA or anti-BBK32 antibodies for use in passive immunization methods for prevention of Borrelia adhesion to Dcn or Fbn, and treatment of infections due to Borrelia invasion, and particularly invasion by B. burgdorferi, sensu strictu and lato. Such anti-DbpA or anti-BBK32 antibodies are also contemplated for use in passive immunization methods for prevention of bacterial adhesion to Dcn or Fbn, and treatment of infections caused by any bacterial species which binds to Dcn or Fbn upon invasion.

2. Recombinant Expression of Db A and BBK32

The present invention also concerns recombinant host cells for expression of one or more isolated dbpA or bbk32 genes. It is contemplated that virtually any host cell may be employed for this purpose, but certain advantages may be found in using a bacterial host cell such as E. coli, S. typhimurium, B. subtilis, or others. Expression in eukaryotic cells is also contemplated such as those derived from yeast, insect, or mammalian cell lines. These recombinant host cells may be employed in connection with “overexpressing” DBPs and FBPs, that is, increasing the level of expression over that found naturally in Borrelia, in particular, B. burgdorferi, sensu strictu and lato, or related spirochete.

Proteins having amino acid sequences derived from, or similar to the DbpA or BBK32 proteins of the present invention are contemplated to have affinity for Dcn or Fbn and may be purified from other constituents of Borrelia, in particular, B. burgdorferi, sensu strictu and lato, or recombinant host cells by chromatography on matrices containing Dcn or Fbn, so-called “affinity chromatography.” DBPs and FBPs may also be purified by methodologies not relying on affinity for Dcn or Fbn such as ion exchange chromatography, size exclusion chromatography, metal chelation chromatography, or the like. Buffer, detergent, and other conditions may be dissimilar from those optimal for “affinity chromatography.” In a preferred embodiment, an affinity matrix comprising Dcn, Fbn or a related proteoglycan may be used for the isolation of DBPs and FBPs from solution, or alternatively, isolation of intact bacteria expressing DBPs or FBPs, or even membrane fragments of bacteria expressing DBPs or FBPs.

A particular aspect of this invention provides novel ways in which to utilize recombinant DBPs or DBP-derived peptides, nucleic acid segments encoding these peptides, recombinant vectors and transformed host cells comprising one or more dbp genes or dbp-derived nucleic acid segments, recombinant vectors and transformed host cells comprising one or more dbp genes or dbp-derived DNA segments, and recombinant vectors and transformed host cells comprising one or more Borrelia dbp-derived nucleic acid segments, in particular one or more dbpA or dbpB nucleic acid segments (as described in U.S. Pat. Nos. 6,228,835; 6,248,517; and 6,214,355 herein incorporated by reference) from B. burgdorferi, sensu strictu and lato. Similarly, a particular aspect of this invention provides novel ways in which to utilize recombinant FBP or FBP-derived peptides, nucleic acid segments encoding these peptides, recombinant vectors and transformed host cells comprising one or more fbp genes or fbp-derived nucleic acid segments, recombinant vectors and transformed host cells comprising one or more fbp genes or fbp-derived DNA segments, and recombinant vectors and transformed host cells comprising one or more Borrelia fbp-derived nucleic acid segments, in particular one or more fbp nucleic acid segments from B. burgdorferi, sensu strictu and lato.

As is well known to those of skill in the art, many such vectors and host cells are readily available, one particular detailed example of a suitable vector for expression in mammalian cells is that described in U.S. Pat. No. 5,168,050, incorporated herein by reference. However, there is no requirement that a highly purified vector be used, so long as the coding segment employed encodes a protein or peptide of interest (e.g., a DbpA or BBK32 protein from Borrelia, and particularly a DbpA or BBK32 protein from B. burgdorferi, sensu strictu and lato), and does not include any coding or regulatory sequences that would have an adverse effect on the cells. Therefore, it will also be understood that useful nucleic acid sequences may include additional residues, such as additional non-coding sequences flanking either of the 5′ or 3′ portions of the coding region or may include various regulatory sequences.

After identifying an appropriate epitope-encoding nucleic acid molecule, it may be inserted into any one of the many vectors currently known in the art, so that it will direct the expression and production of the protein or peptide epitope of interest (e.g., a DbpA or BBK32 protein from Borrelia and in particular, from B. burgdorferi, sensu strictu and lato) when incorporated into a host cell. In a recombinant expression vector, the coding portion of the DNA segment is positioned under the control of a promoter. The promoter may be in the form of the promoter which is naturally associated with a DBP-encoding nucleic acid segment or FBP-encoding nucleic acid segment, as may be obtained by isolating the 5′ non-coding sequences located upstream of the coding segment, for example, using recombinant cloning and/or PCR.TM. technology, in connection with the compositions disclosed herein. Direct amplification of nucleic acids using the PCR.TM. technology of U.S. Pat. Nos. 4,683,195 and 4,683,202 (herein incorporated by reference) are particularly contemplated to be useful in such methodologies.

In certain embodiments, it is contemplated that particular advantages will be gained by positioning one or more DBP-encoding DNA segments or FBP-encoding DNA segments under the control of one or more recombinant, or heterologous, promoters. As used herein, a recombinant or heterologous promoter is intended to refer to a promoter that is not normally associated with a dbpA, bbk32, dbpA or bbk-like gene segment in its natural environment. A “dbp-like gene segment” is intended to mean any nucleic acid segment which hybridizes to a dbpA gene under conditions of moderate to high stringency. A “fbp-like gene segment” is intended to mean any nucleic acid segment which hybridizes to a bbk32 gene under conditions of moderate to high stringency. Such promoters may include those normally associated with other MSCRAMM-encoding genes, and/or promoters isolated from any other bacterial, viral, eukaryotic, or mammalian cell. Naturally, it will be important to employ a promoter that effectively directs the expression of the DNA segment in the particular cell containing the vector comprising one or more DBP- or FBP-encoding nucleic acid segments.

The use of recombinant promoters to achieve protein expression is generally known to those of skill in the art of molecular biology, (see e.g., Sambrook et al., 1989, herein incorporated by reference). The promoters employed may be constitutive, or inducible, and can be used under the appropriate conditions to direct high level or regulated expression of the introduced DNA segment. For eukaryotic expression, the currently preferred promoters are those such as CMV, RSV LTR, the SV40 promoter alone, and the SV40 promoter in combination with the SV40 enhancer. In preferred embodiments, the expression of recombinant DBPs and FBPs may be carried out using prokaryotic expression systems, and in particular bacterial systems such as E. coli. Such prokaryotic expression of nucleic acid segments of the present invention may be performed using methods known to those of skill in the art, and will likely comprise expression vectors and promoter sequences such as those provided by lpp, tac, trp, lac, lacUV5 or T7 promoters. Alternatively, the inventors contemplate that a native or genetically-modified dbp or fbp promoter will also be useful in the construction of recombinant vectors expressing dbpA and/or bbk32 genes.

For the expression of DbpA, BBK32, and DbpA- or BBK32-derived epitopes, once a suitable clone or clones have been obtained, whether they be native sequences or genetically-modified, one may proceed to prepare an expression system for the recombinant preparation of one or more DBP, DBP-derived peptides, FBP, or FBP-derived peptide. The engineering of DNA segment(s) for expression in a prokaryotic or eukaryotic system may be performed by techniques generally known to those of skill in recombinant expression. It is believed that virtually any expression system may be employed in the expression of one or more DBPs, DBP-derived epitopes, FBPs, or FBP-derived epitopes.

Alternatively, it may be desirable in certain embodiments to express one or more DBPs, DBP-derived epitopes, FBPs, or FBP-derived epitopes in eukaryotic expression systems. The DNA sequences encoding the desired DBPs, DBP-derived epitopes, FBPs, or FBP-derived epitopes (either native or mutagenized) may be separately expressed in bacterial systems, with the encoded proteins being expressed as fusion with beta.-galactosidase, ubiquitin, Schistosoma japonicum glutathione S-transferase, S. aureus. Protein A, maltose binding protein, and the like. It is believed that bacterial expression will ultimately have advantages over eukaryotic expression in terms of ease of use and quantity of materials obtained thereby.

It is proposed that transformation of host cells with DNA segments encoding such epitopes will provide a convenient means for obtaining one or more DBPs, DBP-derived peptides, FBPs or FBP-derived peptides. Genomic sequences are suitable for eukaryotic expression, as the host cell will, of course, process the genomic transcripts to yield functional mRNA for translation into protein.

It is similarly believed that almost any eukaryotic expression system may be utilized for the expression of one or more DBPs, DBP-derived epitopes, FBPs and FBP-derived epitopes, e.g., baculovirus-based, glutamine synthase-based or dihydrofolate reductase-based systems may be employed. In preferred embodiments it is contemplated that plasmid vectors incorporating an origin of replication and an efficient eukaryotic promoter, as exemplified by the eukaryotic vectors of the pCMV series, such as pCMV5, will be of most use.

For expression in this manner, one would position the coding sequences adjacent to and under the control of the promoter. It is understood in the art that to bring a coding sequence under the control of such a promoter, one positions the 5′ end of the transcription initiation site of the transcriptional reading frame of the protein between about 1 and about 50 nucleotides “downstream” of (i.e., 3′ of) the chosen promoter.

Where eukaryotic expression is contemplated, one will also typically desire to incorporate into the transcriptional unit which includes nucleic acid sequences encoding one or more DBPs, DBP-derived peptides, FBPs and FBP-derived peptides, an appropriate polyadenylation site (e.g., 5′-AATAAA-3′) if one was not contained within the original cloned segment. Typically, the poly-A addition site is placed about 30 to 2000 nucleotides “downstream” of the termination site of the protein at a position prior to transcription termination.

It is contemplated that virtually any of the commonly employed host cells can be used in connection with the expression of one or more DBPs, DBP-derived epitopes, FBPs and FBP-derived epitopes in accordance herewith. Examples include cell lines typically employed for eukaryotic expression such as 239, AtT-20, HepG2, VERO, HeLa, CHO, WI 38, BHK, COS-7, RIN and MDCK cell lines.

It is further contemplated that a DbpA, BBK32, or epitopic peptides derived from one or more native or recombinant DBPs or FBPs may be “overexpressed”, i.e., expressed in increased levels relative to its natural expression in human cells, or even relative to the expression of other proteins in a recombinant host cell containing a dbp-or fbp-encoding DNA segment. Such overexpression may be assessed by a variety of methods, including radiolabeling and/or protein purification. However, simple and direct methods are preferred, for example, those involving SDS/PAGE and protein staining or Western blotting, followed by quantitative analyses, such as densitometric scanning of the resultant gel or blot. A specific increase in the level of the recombinant protein or peptide in comparison to the level in natural DBP-or FBP-producing cells is indicative of overexpression, as is a relative abundance of the specific protein in relation to the other proteins produced by the host cell and, e.g., visible on a gel.

As used herein, the term “engineered” or “recombinant” cell is intended to refer to a cell into which a recombinant gene, such as a gene encoding a DBP or a FBP has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a single structural gene, an entire genomic clone comprising a structural gene and flanking DNA, or an operon or other functional nucleic acid segment which may also include genes positioned either upstream and/or downstream of the promoter, regulatory elements, or structural gene itself, or even genes not naturally associated with the particular structural gene of interest.

Where the introduction of a recombinant version of one or more of the foregoing genes is required, it will be important to introduce the gene such that it is under the control of a promoter that effectively directs the expression of the gene in the cell type chosen for engineering. In general, one will desire to employ a promoter that allows constitutive (constant) expression of the gene of interest. Commonly used constitutive eukaryotic promoters include viral promoters such as the cytomegalovirus (CMV) promoter, the Rous sarcoma long-terminal repeat (LTR) sequence, or the SV40 early gene promoter. The use of these constitutive promoters will ensure a high, constant level of expression of the introduced genes. The inventors have noticed that the level of expression from the introduced genes of interest can vary in different clones, or genes isolated from different strains or bacteria Thus, the level of expression of a particular recombinant gene can be chosen by evaluating different clones derived from each transfection experiment; once that line is chosen, the constitutive promoter ensures that the desired level of expression is permanently maintained. It may also be possible to use promoters that are specific for cell type used for engineering, such as the insulin promoter in insulinoma cell lines, or the prolactin or growth hormone promoters in anterior pituitary cell lines.

3. Methods for Generating an Immune Response

A further aspect of the invention is the preparation of immunological compositions, and in particular anti-DbpA and anti-BBK32 antibodies for diagnostic and therapeutic methods relating to the detection and treatment of infections caused by B. burgdorferi and related Borreliae including B. burgdoferi sensu strictu and lato.

Also disclosed in a method of generating an immune response in an animal. The method generally involves administering to an animal a pharmaceutical composition comprising an immunologically effective amount of a peptide composition disclosed herein. Preferred peptide compositions include the DbpA peptide disclosed in SEQ ID NO:4; as well as the BBK32 peptide disclosed in SEQ ID NO:2.

Also considered in this invention is an isolated protein of SEQ ID NO:2 or antigenic peptides thereof comprising an amino acid sequence of at least about 10 amino acids residues from SEQ ID NO:2.

Particularly preferred is the isolated protein of SEQ ID NO:2 or antigenic peptides thereof comprising an amino acid sequence of at least about 10 amino acids residues from SEQ ID NO:2, wherein said contiguous amino acid residues are located between residues 125 and 165 of SEQ ID NO:2.

The invention also encompasses DBP, FBP, DBP- and FBP-derived peptide antigen compositions together with pharmaceutically-acceptable excipients, carriers, diluents, adjuvants, and other components, such as additional peptides, antigens, or outer membrane preparations, as may be employed in the formulation of particular vaccines.

The invention also encompasses a method to elicit an immunological response in an animal, said method comprising injecting a composition comprising an isolated fibronectin binding protein and an isolated decorin binding protein into the animal.

Also considered is the method, wherein said isolated fibronectin binding protein has the amino acid sequence of SEQ ID NO:2, and said isolated decorin binding protein has the amino acid sequence of SEQ ID NO:4.

Preferred is the method, wherein said composition comprises an isolated decorin binding protein of SEQ ID NO:4 and an isolated fibronectin binding protein comprising an amino acid sequence of at least about 10 amino acids from SEQ ID NO:2.

Preferred is the method, wherein said composition comprises an isolated decorin binding protein of SEQ ID NO:4 and an isolated fibronectin binding protein comprising an amino acid sequence of at least about 25 amino acids from SEQ ID NO:2.

Preferred is the method, wherein said composition comprises an isolated decorin binding protein of SEQ ID NO:4 and an isolated fibronectin binding protein comprising an amino acid sequence of at least about 50 amino acids from SEQ ID NO:2.

Preferred is the method, wherein said composition comprises an isolated decorin binding protein of SEQ ID NO:4 and an isolated fibronectin binding protein comprising an amino acid sequence of at least about 100 amino acids from SEQ ID NO:2.

Also considered in the invention is the method, wherein said composition comprises an isolated decorin binding protein comprising an amino acid sequence of at least about 10 amino acids from SEQ ID NO:4 and an isolated fibronectin binding protein of SEQ ID NO:2.

Also considered is the method, wherein said composition comprises an isolated decorin binding protein comprising an amino acid sequence of at least about 25 amino acids from SEQ ID NO:4 and an isolated fibronectin binding protein of SEQ ID NO:2.

Also considered is the method, wherein said composition comprises an isolated decorin binding protein comprising an amino acid sequence of at least about 50 contiguous amino acids from SEQ ID NO:4 and an isolated fibronectin binding protein of SEQ ID NO:2.

Also considered is the method, wherein said composition comprises an isolated decorin binding protein comprising an amino acid sequence of at least about 100 amino acids from SEQ ID NO:4 and an isolated fibronectin binding protein of SEQ ID NO:2.

Also considered is the method, wherein said composition comprises an isolated decorin binding protein comprising an amino acid sequence of at least about 10 amino acids from SEQ ID NO:4 and an isolated fibronectin binding protein comprising an amino acid sequence of at least 10 amino acids from SEQ ID NO:2.

Also considered is the method, wherein said composition comprises an isolated decorin binding protein comprising an amino acid sequence of at least about 10 amino acids from SEQ ID NO:4 and an isolated fibronectin binding protein comprising an amino acid sequence of at least 25 amino acids from SEQ ID NO:2.

Also considered is the method, wherein said composition comprises an isolated decorin binding protein comprising an amino acid sequence of at least about 10 amino acids from SEQ ID NO:4 and an isolated fibronectin binding protein comprising an amino acid sequence of at least 50 amino acids from SEQ ID NO:2.

Also considered is the method, wherein said composition comprises an isolated decorin binding protein comprising an amino acid sequence of at least about 10 amino acids from SEQ ID NO:4 and an isolated fibronectin binding protein comprising an amino acid sequence of at least 100 amino acids from SEQ ID NO:2.

Also considered is the method, wherein said composition comprises an isolated decorin binding protein comprising an amino acid sequence of at least about 25 amino acids from SEQ ID NO:4 and an isolated fibronectin binding protein comprising an amino acid sequence of at least 10 amino acids from SEQ ID NO:2.

Also considered is the method, wherein said composition comprises an isolated decorin binding protein comprising an amino acid sequence of at least about 25 amino acids from SEQ ID NO:4 and an isolated fibronectin binding protein comprising an amino acid sequence of at least 25 amino acids from SEQ ID NO:2.

Also considered is the method, wherein said composition comprises an isolated decorin binding protein comprising an amino acid sequence of at least about 25 amino acids from SEQ ID NO:4 and an isolated fibronectin binding protein comprising an amino acid sequence of at least 50 amino acids from SEQ ID NO:2.

Also considered is the method, wherein said composition comprises an isolated decorin binding protein comprising an amino acid sequence of at least about 25 amino acids from SEQ ID NO:4 and an isolated fibronectin binding protein comprising an amino acid sequence of at least 100 amino acids from SEQ ID NO:2.

Also considered is the method, wherein said composition comprises an isolated decorin binding protein comprising an amino acid sequence of at least about 50 amino acids from SEQ D) NO:4 and an isolated fibronectin binding protein comprising an amino acid sequence of at least 10 amino acids from SEQ ID NO:2.

Also considered is the method, wherein said composition comprises an isolated decorin binding protein comprising an amino acid sequence of at least about 50 amino acids from SEQ ID NO:4 and an isolated fibronectin binding protein comprising an amino acid sequence of at least 25 amino acids from SEQ ID NO:2.

Also considered is the method, wherein said composition comprises an isolated decorin binding protein comprising an amino acid sequence of at least about 50 amino acids from SEQ ID NO:4 and an isolated fibronectin binding protein comprising an amino acid sequence of at least 50 amino acids from SEQ ID NO:2.

Also considered is the method, wherein said composition comprises an isolated decorin binding protein comprising an amino acid sequence of at least about 50 amino acids from SEQ ID NO:4 and an isolated fibronectin binding protein comprising an amino acid sequence of at least 100 amino acids from SEQ ID NO:2.

Also considered is the method, wherein said composition comprises an isolated decorin binding protein comprising an amino acid sequence of at least about 100 amino acids from SEQ ID NO:4 and an isolated fibronectin binding protein comprising an amino acid sequence of at least 10 amino acids from SEQ ID NO:2.

Also considered is the method, wherein said composition comprises an isolated decorin binding protein comprising an amino acid sequence of at least about 100 amino acids from SEQ ID NO:4 and an isolated fibronectin binding protein comprising an amino acid sequence of at least 25 amino acids from SEQ ID NO:2.

Also considered is the method, wherein said composition comprises an isolated decorin binding protein comprising an amino acid sequence of at least about 100 amino acids from SEQ ID NO:4 and an isolated fibronectin binding protein comprising an amino acid sequence of at least 50 amino acids from SEQ ID NO:2.

Also considered is the method, wherein said composition comprises an isolated decorin binding protein comprising an amino acid sequence of at least about 100 amino acids from SEQ ID NO:4 and an isolated fibronectin binding protein comprising an amino acid sequence of at least 100 amino acids from SEQ ID NO:2.

The nucleic acid sequences of the present invention encode DBP and FBP and are useful to generate pure recombinant DBP or FBP for administration to a host. Such administration is useful to prevent adherence of Borreliae, and in particular, B. burgdorferi, sensu strictu and lato, to the host's tissues or as a vaccine to induce therapeutic antibodies.

It is shown herein that antisera raised against and reactive with one or more DBPs or FBPs is inhibitory to in vitro and in vivo growth of various Borrelia strains. Thus, it is contemplated that administration of antibodies reactive with one or more DBPs and FBPs to at-risk subjects will be effective for prophylaxis of, and in the case of infected subjects for therapy of, Lyme disease.

Therefore, this invention envision a method for preventing Lyme disease in a animal, comprising administering to the animal an effective amount of vaccine, wherein said vaccine comprises a component of a decorin binding protein and a component of a fibronectin binding protein.

A more preferred method is the method, wherein said decorin binding protein is DbpA and fibronectin binding protein is BBK32.

Also envisioned is the method, wherein said vaccine comprises an isolated decorin binding protein of SEQ ID NO:4 and an isolated fibronectin binding protein comprising an amino acid sequence of at least about 10 amino acids from SEQ ID NO:2.

Also envisioned is the method, wherein said vaccine comprises an isolated decorin binding protein of SEQ ID NO:4 and an isolated fibronectin binding protein comprising an amino acid sequence of at least about 25 amino acids from SEQ ID NO:2.

Also envisioned is the method, wherein said vaccine comprises an isolated decorin binding protein of SEQ ID NO:4 and an isolated fibronectin binding protein comprising an amino acid sequence of at least about 50 amino acids from SEQ ID NO:2.

Also envisioned is the method, wherein said vaccine comprises an isolated decorin binding protein of SEQ ID NO:4 and an isolated fibronectin binding protein comprising an amino acid sequence of at least about 100 amino acids from SEQ ID NO:2.

Also envisioned is the method, wherein said vaccine comprises an isolated decorin binding protein comprising an amino acid sequence of at least about 10 amino acids from SEQ ID NO:4 and an isolated fibronectin binding protein of SEQ ID NO:2.

Also envisioned is the method, wherein said vaccine comprises an isolated decorin binding protein comprising an amino acid sequence of at least about 25 amino acids from SEQ ID NO:4 and an isolated fibronectin binding protein of SEQ ID NO:2.

Also envisioned is the method, wherein said vaccine comprises an isolated decorin binding protein comprising an amino acid sequence of at least about 50 amino acids from SEQ ID NO:4 and an isolated fibronectin binding protein of SEQ ID NO:2.

Also envisioned is the method; wherein said vaccine comprises an isolated decorin binding protein comprising an amino acid sequence of at least about 100 amino acids from SEQ ID NO:4 and an isolated fibronectin binding protein of SEQ ID NO:2.

This invention also encompass the method, wherein said vaccine comprises an isolated decorin binding protein comprising an amino acid sequence of at least about 10 amino acids from SEQ ID NO:4 and an isolated fibronectin binding protein comprising an amino acid sequence of at least 10 amino acids from SEQ ID NO:2.

This invention also encompass the method, wherein said vaccine comprises an isolated decorin binding protein comprising an amino acid sequence of at least about 10 amino acids from SEQ ID NO:4 and an isolated fibronectin binding protein comprising an amino acid sequence of at least 25 amino acids from SEQ ID NO:2.

This invention also encompass the method, wherein said vaccine comprises an isolated decorin binding protein comprising an amino acid sequence of at least about 10 amino acids from SEQ ID NO:4 and an isolated fibronectin binding protein comprising an amino acid sequence of at least 50 amino acids from SEQ ID NO:2.

This invention also encompass the method, wherein said vaccine comprises an isolated decorin binding protein comprising an amino acid sequence of at least about 10 amino acids from SEQ ID NO:4 and an isolated fibronectin binding protein comprising an amino acid sequence of at least 100 amino acids from SEQ ID NO:2.

This invention also encompass the method, wherein said vaccine comprises an isolated decorin binding protein comprising an amino acid sequence of at least about 25 amino acids from SEQ ID NO:4 and an isolated fibronectin binding protein comprising an amino acid sequence of at least 10 amino acids from SEQ ID NO:2.

This invention also encompass the method, wherein said vaccine comprises an isolated decorin binding protein comprising an amino acid sequence of at least about 25 amino acids from SEQ ID NO:4 and an isolated fibronectin binding protein comprising an amino acid sequence of at least 25 amino acids from SEQ ID NO:2.

This invention also encompass the method, wherein said vaccine comprises an isolated decorin binding protein comprising an amino acid sequence of at least about 25 amino acids from SEQ ID NO:4 and an isolated fibronectin binding protein comprising an amino acid sequence of at least 50 amino acids from SEQ ID NO:2.

This invention also encompass the method, wherein said vaccine comprises an isolated decorin binding protein comprising an amino acid sequence of at least about 25 amino acids from SEQ ID NO:4 and an isolated fibronectin binding protein comprising an amino acid sequence of at least 100 amino acids from SEQ ID NO:2.

This invention also encompass the method, wherein said vaccine comprises an isolated decorin binding protein comprising an amino acid sequence of at least about 50 amino acids from SEQ ID NO:4 and an isolated fibronectin binding protein comprising an amino acid sequence of at least 10 amino acids from SEQ ID NO:2.

This invention also encompass the method, wherein said vaccine comprises an isolated decorin binding protein comprising an amino acid sequence of at least about 50 amino acids from SEQ ID NO:4 and an isolated fibronectin binding protein comprising an amino acid sequence of at least 25 amino acids from SEQ ID NO:2.

This invention also encompass the, wherein said vaccine comprises an isolated decorin binding protein comprising an amino acid sequence of at least about 50 amino acids from SEQ ID NO:4 and an isolated fibronectin binding protein comprising an amino acid sequence of at least 50 amino acids from SEQ ID NO:2.

This invention also encompass the method, wherein said vaccine comprises an isolated decorin binding protein comprising an amino acid sequence of at least about 50 amino acids from SEQ ID NO:4 and an isolated fibronectin binding protein comprising an amino acid sequence of at least 100 amino acids from SEQ ID NO:2.

This invention also encompass the method, wherein said vaccine comprises an isolated decorin binding protein comprising an amino acid sequence of at least about 100 amino acids from SEQ ID NO:4 and an isolated fibronectin binding protein comprising an amino acid sequence of at least 10 amino acids from SEQ ID NO:2.

This invention also encompass the method, wherein said vaccine comprises an isolated decorin binding protein comprising an amino acid sequence of at least about 100 amino acids from SEQ ID NO:4 and an isolated fibronectin binding protein comprising an amino acid sequence of at least 25 amino acids from SEQ ID NO:2.

This invention also encompass the method, wherein said vaccine comprises an isolated decorin binding protein comprising an amino acid sequence of at least about 100 amino acids from SEQ ID NO:4 and an isolated fibronectin binding protein comprising an amino acid sequence of at least 50 amino acids from SEQ ID NO:2.

This invention also encompass the method, wherein said vaccine comprises an isolated decorin binding protein comprising an amino acid sequence of at least about 100 amino acids from SEQ ID NO:4 and an isolated fibronectin binding protein comprising an amino acid sequence of at least 100 amino acids from SEQ ID NO:2.

Antibodies may be of several types including those raised in heterologous donor animals or human volunteers immunized with DBPs and/or FBPs, monoclonal antibodies (mAbs) resulting from hybridomas derived from fusions of B cells from DBP- or FBP-immunized animals or humans with compatible myeloma cell lines, so-called “humanized” mAbs resulting from expression of gene fusions of combinatorial determining regions of mAb-encoding genes from heterologous species with genes encoding human antibodies, or DBP- or FBP-reactive antibody-containing fractions of plasma from human donors residing in Lyme disease-endemic areas. It is contemplated that any of the techniques described above might be used for the vaccination of subjects for the purpose of antibody production. Optimal dosing of such antibodies is highly dependent upon the pharmacokinetics of the specific antibody population in the particular species to be treated, but it is anticipated that it will be necessary to maintain in these subjects a serum concentration of DBP- or FBP-reactive antibodies that is at least twice that required for inhibition of in vitro growth of endemic Borrelia strains. It is contemplated that the duration of dosing maintaining anti-DbpA and or anti-BBK32 levels at these inhibitory antibody concentrations would be for at least four to eight weeks following presumptive exposure to a Borrelia, and in particular, B. burgdorferi, or throughout the duration of symptoms of Lyme disease and for at least four to eight weeks after cessation of these symptoms.

Using the peptide antigens described herein, the present invention also provides methods of generating an immune response, which methods generally comprise administering to an animal, a pharmaceutically-acceptable composition comprising an immunologically effective amount of one or more DBP and/or FBP peptide compositions. Preferred animals include mammals, and particularly humans. Other preferred animals include murines, bovines, equines, porcines, canines, and felines. The composition may include partially or significantly purified DBP and/or FBP epitopes, obtained from natural or recombinant sources, which proteins or peptides may be obtainable naturally or either chemically synthesized, or alternatively produced in vitro from recombinant host cells expressing DNA segments encoding such epitopes. Smaller peptides that include reactive epitopes, such as those between about 10 and about 50, or even between about 50 and about 100 amino acids in length will often be preferred. The antigenic proteins or peptides may also be combined with other agents, such as other Borrelia peptide or nucleic acid compositions, if desired.

By “effective amount” is meant an amount of a peptide composition that is capable of generating an immune response in the recipient animal. This includes both the generation of an antibody response (B cell response), and/or the stimulation of a cytotoxic immune response (T cell response). The generation of such an immune response will have utility in both the production of useful bioreagents, e.g., CTLs and, more particularly, reactive antibodies, for use in diagnostic embodiments, and will also have utility in various prophylactic or therapeutic embodiments. Therefore, although these methods for the stimulation of an immune response include vaccination regimens designed to prevent or lessen significant infections caused by Borrelias or other bacteria expressing a DBP and FBP, and treatment regimens that may lessen the severity or duration of any infection, it will be understood that achieving either of these end results is not necessary for practicing these aspects of the invention. Such treatment methods may be used particularly for the treatment of infections caused by pathogens such as B. burgdorferi, sensu strictu and lato, related Borreliae species, and other bacteria which express one or more DBPs and FBPs and in particular DbpA and/or BBK32 and adhere to Dcn or Fbn.

Further means contemplated by the inventors for generating an immune response in an animal includes administering to the animal, or human subject, a pharmaceutically-acceptable composition comprising an immunologically effective amount of a nucleic acid composition encoding a DBP or FBP epitope, or an immunologically effective amount of an attenuated live organism that includes and expresses such a nucleic acid composition. The “immunologically effective amounts” are those amounts capable of stimulating a B-cell and/or T-cell response.

Immunoformulations of this invention, whether intended for vaccination, treatment, or for the generation of antibodies useful in the detection of Borrelias and in particular B. burgdorferi, the prevention of bacterial adhesion, or in the case of bacterial colonization, promotion of bacterial adhesion to ECM components such as Dcn and Fbn, may comprise native, or synthetically-derived antigenic peptide fragments from these proteins. As such, antigenic functional equivalents of the proteins and peptides described herein also fall within the scope of the present invention. An “antigenically functional equivalent” protein or peptide is one that incorporates an epitope that is immunologically cross-reactive with one or more epitopes derived from any of the particular MSCRAMM proteins disclosed (e.g., DBPs or FBPs), and particularly the DBP and FBP of B. burgdorferi. Antigenically functional equivalents, or epitopic sequences, may be first designed or predicted and then tested, or may simply be directly tested for cross-reactivity.

The identification or design of suitable DBP and FBP epitopes, and/or their functional equivalents, suitable for use in immunoformulations, vaccines, or simply as antigens (e.g., for use in detection protocols), is a relatively straightforward matter. For example, one may employ the methods of Hopp, as enabled in U.S. Pat. No. 4,554,101, incorporated herein by reference, that teaches the identification and preparation of epitopes from amino acid sequences on the basis of hydrophilicity. The methods described in several other papers, and software programs based thereon, can also be used to identity epitopic core sequences. For example, Chou and Fasman (1974a,b; 1978a,b; 1979); Jameson and Wolf (1988); Wolf et al. (1988); and Kyte and Doolittle (1982) all address this subject. The amino acid sequence of these “epitopic core sequences” may then be readily incorporated into peptides, either through the application of peptide synthesis or recombinant technology.

It is proposed that the use of shorter antigenic peptides, e.g., about 25 to about 50, or even about 10 to 25 amino acids in length, that incorporate epitopes of one or more DBPs or FBPs will provide advantages in certain circumstances, for example, in the preparation of vaccines or in immunologic detection assays. Exemplary advantages include the ease of preparation and purification, the relatively low cost and improved reproducibility of production, and advantageous biodistribution.

In still further embodiments, the present invention concerns immunodetection methods and associated kits. It is contemplated that the proteins or peptides of the invention may be employed to detect antibodies having reactivity therewith, or, alternatively, antibodies prepared in accordance with the present invention, may be employed to detect DBPs, DBP-derived peptides, FBPs, or FBP-derived peptides. Either type of kit may be used in the immunodetection of compounds, present within clinical samples, that are indicative of Lyme disease or related infections caused by Borreliae, and in particular B. burgdorferi. The kits may also be used in antigen or antibody purification, as appropriate.

In general, the preferred immunodetection methods will include first obtaining a sample suspected of containing a DBP-reactive antibody or a FBP-reactive antibody, such as a biological sample from a patient, and contacting the sample with a first DBP, FBP or peptide under conditions effective to allow the formation of an immunocomplex (primary immune complex). One then detects the presence of any primary immunocomplexes that are formed.

Contacting the chosen sample with the DBP, FBP or peptide under conditions effective to allow the formation of (primary) immune complexes is generally a matter of simply adding the protein or peptide composition to the sample. One then incubates the mixture for a period of time sufficient to allow the added antigens to form immune complexes with, i.e., to bind to, any antibodies present within the sample. After this time, the sample composition, such as a tissue section, ELISA plate, dot blot or western blot, will generally be washed to remove any non-specifically bound antigen species, allowing only those specifically bound species within the immune complexes to be detected.

The detection of immunocomplex formation is well known in the art and may be achieved through the application of numerous approaches known to the skilled artisan and described in various publications, such as, e.g., Nakamura et al. (1987), incorporated herein by reference. Detection of primary immune complexes is generally based upon the detection of a label or marker, such as a radioactive, fluorescent, biological or enzymatic label, with enzyme tags such as alkaline phosphatase, urease, horseradish peroxidase and glucose oxidase being suitable. The particular antigen employed may itself be linked to a detectable label, wherein one would then simply detect this label, thereby allowing the amount of bound antigen present in the composition to be determined.

Alternatively, the primary immune complexes may be detected by means of a second binding ligand that is linked to a detectable label and that has binding affinity for the first protein or peptide. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies and the remaining bound label is then detected.

For diagnostic purposes, it is proposed that virtually any sample suspected of containing the antibodies of interest may be employed. Exemplary samples include clinical samples obtained from a patient such as blood or serum samples, cerebrospinal, synovial, or bronchoalveolar fluid, ear swabs, sputum samples, middle ear fluid or even perhaps urine samples may be employed. This allows for the diagnosis of Lyme disease and related infections caused by Borrelias, and in particular, B. burgdoiferi. Furthermore, it is contemplated that such embodiments may have application to non-clinical samples, such as in the titering of antibody samples, in the selection of hybridomas, and the like. Alternatively, the clinical samples may be from veterinary sources and may include such domestic animals as cattle, sheep, and goats. Samples from feline, canine, and equine sources may also be used in accordance with the methods described herein.

In related embodiments, the present invention contemplates the preparation of kits that may be employed to detect the presence of DBP- or FBP-specific antibodies in a sample. Generally speaking, kits in accordance with the present invention will include a suitable protein or peptide together with an immunodetection reagent, and a means for containing the protein or peptide and reagent.

The immunodetection reagent will typically comprise a label associated with a DBP, a FBP or a peptide, or associated with a secondary binding ligand. Exemplary ligands might include a secondary antibody directed against the first DBP or peptide or antibody, or a biotin or avidin (or streptavidin) ligand having an associated label. Detectable labels linked to antibodies that have binding affinity for a human antibody are also contemplated, e.g., for protocols where the first reagent is a DBP or FBP peptide that is used to bind to a reactive antibody from a human sample. Of course, as noted above, a number of exemplary labels are known in the art and all such labels may be employed in connection with the present invention. The kits may contain antigen or antibody-label conjugates either in fully conjugated form, in the form of intermediates, or as separate moieties to be conjugated by the user of the kit.

The container means will generally include at least one vial, test tube, flask, bottle, syringe or other container means, into which the antigen may be placed, and preferably suitably allocated. Where a second binding ligand is provided, the kit will also generally contain a second vial or other container into which this ligand or antibody may be placed. The kits of the present invention will also typically include a means for containing the vials in close confinement for commercial sale, such as, e.g., injection or blow-molded plastic containers into which the desired vials are retained.

4. Methods for Inhibiting Bacterial Adhesion to decorin (Dcn) and fibronectin (Fbn)

In addition, the DBP and FBP are useful as agents to block B. burgdorferi adherence to Dcn, and proteoglycans which are structurally similar to Dcn such as Laminin, as well as Fbn. In a preferred embodiment of the invention, a therapeutically effective dose of one or more DBPs in combination with one or more FBPs, and in particular one or more DbpA proteins, in combination with BBK32, is administered to a subject to prevent or block adhesion of B. burgdorferi to the host's tissues by conventional methods. The composition is preferably systemically administered, but may be applied topically, e.g., to a localized lesion. The term “therapeutically effective dose” means that amount of a DBP and FBP composition which is sufficient to lessen or prevent adherence of B. burgdorferi to a subject or to neutralize the known deleterious effects of B. burgdorferi infection and may be determined by known clinical methods. Absent adhesion of the bacteria to the tissues, the disease-inducing effects of the microorganism are halted, thus the compositions of the present invention is useful as a therapeutic agent to prevent adhesion of B. burgdorferi and thereby lessen or prevent disease induced by this microorganism.

5. Anti-DbpA and Anti-BBK32 Antibody Compositions

In a preferred embodiment, administration of a therapeutically effective dose of DbpA and/or BBK32 to a subject induces in the subject antibodies which bind and neutralize a Borrelia bacterium (and particularly B. burgdorferi, sensu strictu and lato and related Borreliae), present in the subject, thereby preventing the deleterious effects of this microorganism. Alternatively, anti-Borrelia antibodies, and in particular, anti-B. burgdorferi, sensu strictu and lato and related Borreliae antibodies generated in a first host animal provide antibodies which can be administered to a second subject for passive immunization or treatment against B. burgdorferi sensu strictu and lato infection. Such anti-Borrelia antibodies are also useful as a diagnostic screen for the presence of Borrelias, and in particular B. burgdorferi, sensu strictu and lato or related Borreliae in a test sample, using conventional immunoassay techniques.

In certain aspects, the present invention concerns novel antibody compositions which inhibit Dcn and Fbn binding to bacteria In particular, antibodies to native and synthetically-modified epitopes from DBPs and FBPs have been developed which inhibit Dcn binding to DBPs, and Fbn binding to FBPs, respectively, both in vitro and in vivo. In particular, proteins, peptides and peptide epitopes have been produced to provide vaccine compositions useful in the prevention of Lyme disease and antibody compositions useful in the prevention of Dcn and Fbn binding to Borrelias.

In one aspect, the invention discloses an antibody that interacts with a DBP domain of a bacteria dbp gene product, and particularly, a DBP domain of a B. burgdorferi dbp gene product. In another aspect, the invention discloses an antibody that interacts with a domain of a bacteria fbp gene product, and particularly, a fbp domain of a B. burgdorferi BBK32 gene product. Such antibody may be monoclonal, or preferably polyclonal. In another aspect, the invention discloses an antibody which inhibits bacterial adhesion, and the binding of the gene product to Dcn. Similarly, the invention discloses an antibody which inhibits bacterial adhesion, and the binding of the gene product to Fbn.

Also disclosed is a method for detecting a bacterium expressing a DBP and/or FBP in a sample. The method generally involves obtaining a sample suspected of containing a bacterium expressing such a protein, then contacting the sample with an antibody composition disclosed herein, and detecting the formation of immune complexes. In preferred embodiments, the bacterium is a Borrelia, and most preferably, a B. burgdorferi sensu strictu and lato strain.

6. Methods for Inhibiting Bacterial Colonization

Other aspects of the invention include methods of inhibiting bacterial colonization, and particularly colonization by Borrelias, in an animal by administering to the animal an antibody of the present invention which prevents or significantly reduces the binding of Dcn to the DBP and Fbn to the FBP expressed by the bacteria. Administration of the antibody composition may be prophylactically prior to and/or following diagnosis of Lyme disease or other multisystemic disorders caused by Borrelioses which may involve the skin, joints, heart, and central nervous system. The administration may also be made in passive immunization protocols designed to prevent and/or ameliorate systemic infections by susceptible pathogens, and in particular, to ameliorate the effects of infections by pathogenic B. burgdorferi.

7. Vaccine Formulation and Compositions

It is expected that to achieve an “immunologically effective formulation” it may be desirable to administer DBPs and FBPs to the human or animal subject in a pharmaceutically acceptable composition comprising an immunologically effective amount of DBPs and FBPs mixed with other excipients, carriers, or diluents which may improve or otherwise alter stimulation of B cell and/or T cell responses, or immunologically inert salts, organic acids and bases, carbohydrates, and the like, which promote stability of such mixtures. Immunostimulatory excipients, often referred to as adjuvants, may include salts of aluminum (often referred to as Alums), simple or complex fatty acids and sterol compounds, physiologically acceptable oils, polymeric carbohydrates, chemically or genetically modified protein toxins, and various particulate or emulsified combinations thereof DBPs and FBPs or peptides within these mixtures, or each variant if more than one are present, would be expected to comprise about 0.0001 to 1.0 milligrams, or more preferably about 0.001 to 0.1 milligrams, or even more preferably less than 0.1 milligrams per dose.

Particularly, this invention envisions the use of a composition comprising an isolated decorin binding protein and an isolated fibronectin binding protein.

More particularly, this invention envisions the use of a composition wherein said decorin binding protein has the amino acid sequence of SEQ ID NO:4 and said fibronectin binding protein has the amino acid sequence of SEQ ID NO:2.

Preferred is a composition comprising an isolated decorin binding protein of SEQ ID NO:4 and an isolated fibronectin binding protein comprising an amino acid sequence of at least about 10 amino acids from SEQ ID NO:2.

Also preferred, is a composition comprising an isolated decorin binding protein of SEQ ID NO:4 and an isolated fibronectin binding protein comprising an amino acid sequence of at least about 25 amino acids from SEQ ID NO:2.

Also preferred, is a composition comprising an isolated decorin binding protein of SEQ ID NO:4 and an isolated fibronectin binding protein comprising an amino acid sequence of at least about 50 amino acids from SEQ ID NO:2.

Also preferred, is a composition comprising an isolated decorin binding protein of SEQ ID NO:4 and an isolated fibronectin binding protein comprising an amino acid sequence of at least about 100 amino acids from SEQ ID NO:2.

Similarly envisioned is a composition comprising an isolated decorin binding protein comprising an amino acid sequence of at least about 10 amino acids from SEQ ID NO:4 and an isolated fibronectin binding protein of SEQ ID NO:2.

Similarly envisioned is a composition of claim 7, comprising an isolated decorin binding protein comprising an amino acid sequence of at least about 25 amino acids from SEQ ID NO:4 and an isolated fibronectin binding protein of SEQ ID NO:2.

Similarly envisioned is a composition of comprising an isolated decorin binding protein comprising an amino acid sequence of at least about 50 amino acids from SEQ ID NO:4 and an isolated fibronectin binding protein of SEQ ID NO:2.

Similarly envisioned is a composition comprising an isolated decorin binding protein comprising an amino acid sequence of at least about 100 amino acids from SEQ ID NO:4 and an isolated fibronectin binding protein of SEQ ID NO:2.

Also envisioned in this invention is a composition comprising an isolated decorin binding protein comprising an amino acid sequence of at least about 10 amino acids from SEQ ID NO:4 and an isolated fibronectin binding protein comprising an amino acid sequence of at least 10 amino acids from SEQ ID NO:2.

Similarly envisioned is a composition comprising an isolated decorin binding protein comprising an amino acid sequence of at least about 10 amino acids from SEQ ID NO:4 and an isolated fibronectin binding protein comprising an amino acid sequence of at least 25 amino acids from SEQ ID NO:2.

Similarly envisioned is a composition comprising an isolated decorin binding protein comprising an amino acid sequence of at least about 10 amino acids from SEQ ID NO:4 and an isolated fibronectin binding protein comprising an amino acid sequence of at least 50 amino acids from SEQ ID NO:2.

Similarly envisioned is a composition comprising an isolated decorin binding protein comprising an amino acid sequence of at least about 10 amino acids from SEQ ID NO:4 and an isolated fibronectin binding protein comprising an amino acid sequence of at least 100 amino acids from SEQ ID NO:2.

Also envisioned is a composition comprising an isolated decorin binding protein comprising an amino acid sequence of at least about 25 amino acids from SEQ ID NO:4 and an isolated fibronectin binding protein comprising an amino acid sequence of at least 10 amino acids from SEQ ID NO:2.

Also envisioned is a composition comprising an isolated decorin binding protein comprising an amino acid sequence of at least about 25 amino acids from SEQ ID NO:4 and an isolated fibronectin binding protein comprising an amino acid sequence of at least 25 amino acids from SEQ ID NO:2.

Also envisioned is a composition comprising an isolated decorin binding protein comprising an amino acid sequence of at least about 25 amino acids from SEQ ID NO:4 and an isolated fibronectin binding protein comprising an amino acid sequence of at least 50 amino acids from SEQ ID NO:2.

Also envisioned is a composition comprising an isolated decorin binding protein comprising an amino acid sequence of at least about 25 amino acids from SEQ ID NO:4 and an isolated fibronectin binding protein comprising an amino acid sequence of at least 100 amino acids from SEQ ID NO:2.

Also envisioned is a composition comprising an isolated decorin binding protein comprising an amino acid sequence of at least about 50 amino acids from SEQ ID NO:4 and an isolated fibronectin binding protein comprising an amino acid sequence of at least 10 amino acids from SEQ ID NO:2.

Also envisioned is a composition comprising an isolated decorin binding protein comprising an amino acid sequence of at least about 50 amino acids from SEQ ID NO:4 and an isolated fibronectin binding protein comprising an amino acid sequence of at least 25 amino acids from SEQ ID NO:2.

Also envisioned is a composition comprising an isolated decorin binding protein comprising an amino acid sequence of at least about 50 amino acids from SEQ ID NO:4 and an isolated fibronectin binding protein comprising an amino acid sequence of at least 50 amino acids from SEQ ID NO:2.

Also envisioned is a composition comprising an isolated decorin binding protein comprising an amino acid sequence of at least about 50 amino acids from SEQ ID NO:4 and an isolated fibronectin binding protein comprising an amino acid sequence of at least 100 amino acids from SEQ ID NO:2.

Also envisioned is a composition comprising an isolated decorin binding protein comprising an amino acid sequence of at least about 100 amino acids from SEQ ID NO:4 and an isolated fibronectin binding protein comprising an amino acid sequence of at least 10 amino acids from SEQ ID NO:2.

Also envisioned is a composition comprising an isolated decorin binding protein comprising an amino acid sequence of at least about 100 amino acids from SEQ ID NO:4 and an isolated fibronectin binding protein comprising an amino acid sequence of at least 25 amino acids from SEQ ID NO:2.

Also envisioned is a composition comprising an isolated decorin binding protein comprising an amino acid sequence of at least about 100 amino acids from SEQ ID NO:4 and an isolated fibronectin binding protein comprising an amino acid sequence of at least 50 amino acids from SEQ ID NO:2.

Also envisioned is a composition comprising an isolated decorin binding protein comprising an amino acid sequence of at least about 100 amino acids from SEQ ID NO:4 and an isolated fibronectin binding protein comprising an amino acid sequence of at least 100 amino acids from SEQ D NO:2.

Also contemplated by the present invention is a composition comprising a decorin binding protein of amino acid sequence SEQ ID NO:4 and a fibronectin binding protein of amino acid sequence SEQ ID NO:2, dispersed in a pharmaceutically acceptable carrier or diluent.

The above-mentioned composition further comprising one or more compounds selected from the group consisting of excipients and adjuvants is also considered.

It is also contemplated that attenuated organisms may be engineered to express recombinant dbp or fbp gene products and themselves be delivery vehicles for the invention. Particularly preferred are attenuated bacterial species such as Mycobacterium, and in particular M. bovis, M. smegmatis, or BCG. Alternatively, pox-, polio-, adeno-, or other viruses, and bacteria such as Salmonella, Shigella, Listeria, Streptococcus species may also be used in conjunction with the methods and compositions disclosed herein.

The naked DNA technology, often referred to as genetic immunization, has been shown to be suitable for protection against infectious organisms. Such DNA segments could be used in a variety of forms including naked DNA and plasmid DNA, and may administered to the subject in a variety of ways including parenteral, mucosal, and so-called microprojectile-based “gene-gun” inoculations. The use of dbp and fbp nucleic acid compositions of the present invention in such immunization techniques is thus proposed to be useful as a vaccination strategy against Lyme disease.

It is recognized by those skilled in the art that an optimal dosing schedule of a vaccination regimen may include as many as five to six, but preferably three to five, or even more preferably one to three administrations of the immunizing entity given at intervals of as few as two to four weeks, to as long as five to ten years, or occasionally at even longer intervals.

8. Compositions Comprising Transformed Host Cells and Recombinant Vectors

Particular aspects of the invention concern the use of plasmid vectors for the cloning and expression of recombinant peptides, and particular peptide epitopes comprising either native, or site-specifically mutated DBP or FBP epitopes. The generation of recombinant vectors, transformation of host cells, and expression of recombinant proteins is well known to those of skill in the art. Prokaryotic hosts are preferred for expression of the peptide compositions of the present invention. An example of a preferred prokaryotic host is E. coli, and in particular, E. coli strains ATCC69791, BL21(DE3), JMO101, XL1-Blue.TM., RR1, LE392, B, .chi.sup.1776 (ATCC No. 31537), and W3110 (F.sup.-, .lambda.sup.-, prototrophic, ATCC273325). Alternatively, other Enterobacteriaceae species such as Salmonella typhimurium and Serratia marcescens, or even other Gram-negative hosts including various Pseudomonas species may be used in the recombinant expression of the genetic constructs disclosed herein. Borreliae themselves may be used to express these constructs, and in particular, B. burgdoreri, sensu strictu and lato.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli may be typically transformed using vectors such as pBR322, or any of its derivatives (Bolivar et al., 1977). pBR322 contains genes for ampicillin and tetracycline resistance and thus provides easy means for identifying transformed cells. pBR322, its derivatives, or other microbial plasmids or bacteriophage may also contain, or be modified to contain, promoters which can be used by the microbial organism for expression of endogenous proteins. A preferred vector for cloning the dbp and fbp constructs is pBlueScript.TM., and in particular the construct BG26:pB/2.5(5), or alternatively, vectors based on the pET vector series Novagen, Inc., Madison, Wis.).

In addition, phage vectors containing replicon and control sequences that are compatible with the host microorganism can be used as transforming vectors in connection with these hosts. For example, bacteriophage such as lambda.GEM.TM.-1 may be utilized in making a recombinant vector which can be used to transform susceptible host cells such as E. coli LE392.

Those promoters most commonly used in recombinant DNA construction include the .beta-lactamase (penicillinase) and lactose promoter systems (Chang et al., 1978; Itakura et al., 1977; Goeddel et al., 1979) or the tryptophan (trp) promoter system (Goeddel et al., 1980). The use of recombinant and native microbial promoters is well-known to those of skill in the art, and details concerning their nucleotide sequences and specific methodologies are in the public domain, enabling a skilled worker to construct particular recombinant vectors and expression systems for the purpose of producing compositions of the present invention.

In addition to the preferred embodiment expression in prokaryotes, eukaryotic microbes, such as yeast cultures may also be used in conjunction with the methods disclosed herein. Saccharomyces cerevisiae, or common bakers' yeast is the most commonly used among eukaryotic microorganisms, although a number of other species may also be employed for such eukaryotic expression systems. For expression in Saccharomyces, the plasmid YRp7, for example, is commonly used (Stinchcomb et al., 1979; Kingsman et al., 1979; Tschemper et al., 1980). This plasmid already contains the trpL gene which provides a selection marker for a mutant strain of yeast lacking the ability to grow in tryptophan, for example ATCC No. 44076 or PEP4-1 (Jones, 1977). The presence of the trpL lesion as a characteristic of the yeast host cell genome then provides an effective environment for detecting transformation by growth in the absence of tryptophan.

Suitable promoting sequences in yeast vectors include the promoters for 3-phosphoglycerate kinase (Hitzeman et al., 1980) or other glycolytic enzymes (Hess et al., 1968; Holland et al., 1978), such as enolase, glyceraldehyde-3-phosphate dehydrogenase, hexokinase, pyruvate decarboxylase, phosphofructolinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the MRNA and termination. Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, an origin of replication, and termination sequences is suitable.

In addition to microorganisms, cultures of cells derived from multicellular organisms may also be used as hosts in the routine practice of the disclosed methods. In principle, any such cell culture is workable, whether from vertebrate or invertebrate culture. However, interest has been greatest in vertebrate cells, and propagation of vertebrate cells in culture (tissue culture) has become a routine procedure in recent years. Examples of such useful host cell lines are VERO and HeLa cells, Chinese hamster ovary (CHO) cell lines, and W138, BHK, COS-7, 293 and MDCK cell lines. Expression vectors for such cells ordinarily include (if necessary) an origin of replication, a promoter located in front of the gene to be expressed, along with any necessary ribosome binding sites, RNA splice sites, polyadenylation site, and transcriptional terminator sequences.

For use in mammalian cells, the control functions on the expression vectors are often provided by viral material. For example, commonly used promoters are derived from polyoma, Adenovirus 2, and most frequently Simian Virus 40 (SV40). The early and late promoters of SV40 virus are particularly useful because both are obtained easily from the virus as a fragment which also contains the SV40 viral origin of replication (Fiers et al., 1978). Smaller or larger SV40 fragments may also be used, provided there is included the approximately 250 bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

It will be further understood that certain of the polypeptides may be present in quantities below the detection limits of the Coomassie brilliant blue staining procedure usually employed in the analysis of SDS/PAGE gels, or that their presence may be masked by an inactive polypeptide of similar M.sub.r. Although not necessary to the routine practice of the present invention, it is contemplated that other detection techniques may be employed advantageously in the visualization of particular polypeptides of interest. Immunologically-based techniques such as Western blotting using enzymatically-, radiolabel-, or fluorescently-tagged antibodies described herein are considered to be of particular use in this regard. Alternatively, the peptides of the present invention may be detected by using antibodies of the present invention in combination with secondary antibodies having affinity for such primary antibodies. This secondary antibody may be enzymatically- or radiolabeled, or alternatively, fluorescently-, or colloidal gold-tagged. Means for the labeling and detection of such two-step secondary antibody techniques are well-known to those of skill in the art.

### EXAMPLES

### Example 1

Expression and Purification of the Proteins Needed for Vaccination

TABLE I
Primers Used in the Cloning of bbk32 and SdrF

| Ampli- | | Vector |
|---|---|---|
| fied | | Desti- |
| DNA | Primer Sequence | nation |
| bbk32 | Fwrd | pQE30 |
| | GCGGGATCCGATTTATTCATAAGATATGAAATGAAA | |
| | Rev | pQE30 |
| | GCGAAGCTTGTACCAAACGCATTCTTGTCAATGAT | |
| SdrF | Fwrd | pQE30 |
| | CCCGGATCCGCTGAAGACAATCAATTAG | |
| | Rev | pQE30 |
| | CCCAAGCTTAATTATCCCCCTGTGCTG | |

Recombinant SdrF (used as a control) and BBK32 in the pQE30 vector (His-tag) were expressed in E. coli (JM101) harboring the appropriate plasmid. E. coli was grown in LB until it reached an A₆₀₀ of 0.6. Isopropyl-β-D-thiogalactopyranoside (IPTG) (Life Technologies) was added to a final concentration of 0.2 mM, and the cells were incubated at 37° C. for an additional 4 hours. Cells from a 1 L culture were harvested by centrifugation and resuspended in 10 ml “binding buffer” (BB) (20 mM Tris HCl, 0.5 M NaCl, 15 mM imidazole, pH 8.0) and lysed in a French pressure cell at 11,000 pounds/inch². The lysate was centrifuged at 40,000×g for 15 min and the supernatant filtered through a 0.45 μm filter. A 1 ml iminodiacetic acid Sepharose column (Sigma, St. Louis, Mo.) was charged with 75 mM NiCl₂-6H₂O and equilibrated with BB. The filtered supernatant was applied to the column and washed with 10 volumes of BB, then 10 volumes of BB containing 60 mM imidazole. The bound proteins were eluted with BB containing 200 mM imidazole, dialyzed against PBS containing 10 mM EDTA, then dialyzed against PBS. The protein concentration was determined by the Bicinchionic Acid (BCA) Protein Assay (Pierce) and proteins were stored at −20° C.

### Example 2

Several Adhesins are Involved in the Borrelia Infection Process. zRecently, our laboratory has demonstrated that decorin deficient mice are more resistant to Borrelia infection compared to wild-type controls (Brown et al. 2001). This increased but incomplete resistance to Borrelia infection in these mice suggested that the DBP-decorin interaction(s) play a role in establishment of disease but that other adhesins also may be involved in the disease process.

Material and Methods:

Mice. Specific pathogen-free decorin deficient mice (B1/Swiss×129Sv) (MTV⁻) were provided by Renato Iozzo, Thomas Jefferson University, Philadelphia, Pa.) and back crossed 5 and 10 generations into BALB/c mice (Harlan Sprague Dawley, Indianapolis, Ind.) (Danielson et al. 1997). The animals were maintained in facilities approved by the American Association for Accreditation of Laboratory Animal Care in accordance with current regulations and standards of the United States Department of Agriculture, Department of Health and Human Services, and National Institutes of Health. All animal procedures were approved by the Institutional Animal Care and Use Committee. Female mice were 8-10 weeks old at the start of each experiment.

Bacterial Strains, Culture and Materials. Low-passage B. burgdorferi strain B31 (passage 5) was used in this study and cultured in BSK II (Barbour-Stoenner-Kelly) medium at 34° C. (Barbour et al. 1984). Bacterial cultures were incubated in CO₂-enriched atmosphere-in a GasPak chamber (3BL, Baltimore, Md.) containing BBL GasPak Plus envelopes and a GasPak anaerobic indicator (Beckton Dickinson, Cockeysville, Md.) until the cells reached log phase. The density of bacteria was determined using dark field microscopy and a Petroff-Hausser chamber. E. coli strain JM11 (Qiagen, Chatsworth, Calif.) were grown at 37° C. in Lennox broth (LB) (Difco, Detroit, Mi., containing the appropriate antibiotics.

Vaccinations. Decorin-deficient and wild-type mice were immunized with BBK32, SdrF in complete Freund's adjuvant or with adjuvant alone as described previously (Hanson et al. 1998).

Borrelia Infections. Needle inoculation was performed by injecting 10⁴ B. burgdorferi (100 μl volume) i.d. into shaved dorsal skin at the base of the tail. Borrelia was grown in BSK-II medium with antibiotics (50 μg/ml rifampicin and 100 μg/ml phosphomycin) at 34° C. as described previously (Barbour et al. 1984). Bacterial cultures were incubated in CO₂-enriched atmosphere in a GasPak chamber (BBL, Baltimore, Md.) containing BBL GasPak Plus envelopes and a GasPak anaerobic indicator (Beckton Dickinson, Cockeysville, Md.) until the cells reached log phase. Borrelia were counted using dark field microscopy and a Petroff-Hausser chamber. Culturing of Tissues. Two weeks post infection, ear, bladder, and one joint devoid of skin were harvested under a laminar flow biosafety containment hood. Tissues were cultured in BSK II media and incubated at 34° C. The cultures were checked for the presence of spirochetes 2 weeks later.

Results:

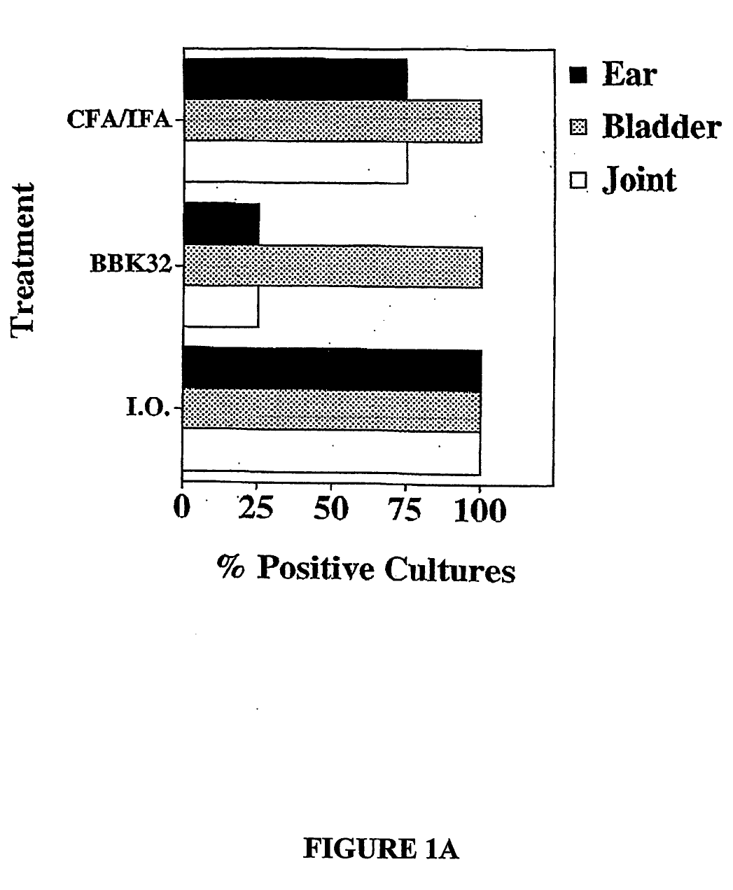

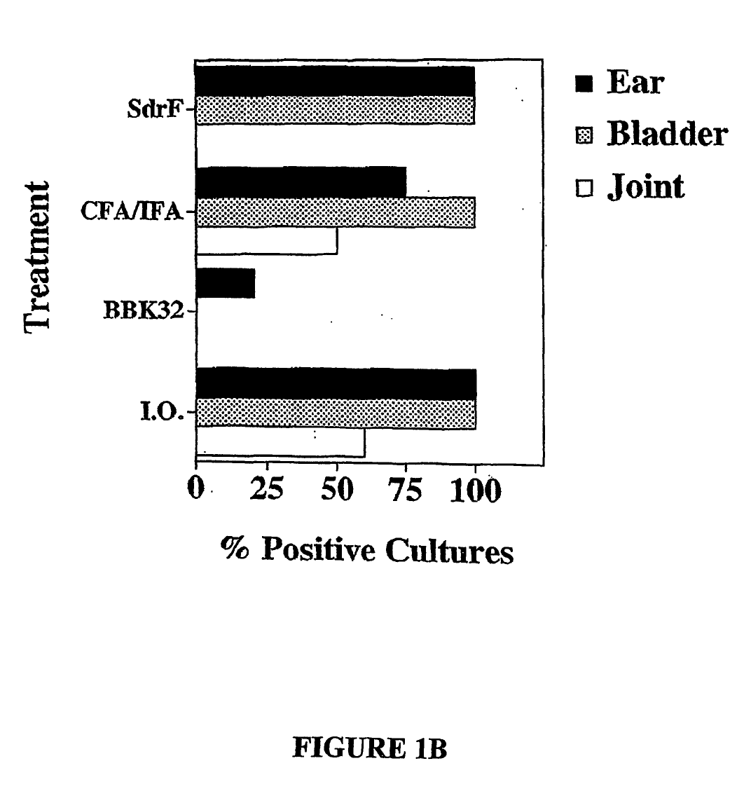

Recombinant BBK32 or the control protein SdrF from Staphylococcus aureus were used to vaccinate decorin-deficient or wild-type mice against the Lyme spirochete Borrelia burgdorferi. Two weeks after the second immunization, mice were infected i.d. with 10⁴ spirochetes at the base of the tail. Two weeks later, the mice were sacrificed and the ear, bladder, and one joint were examined for the presence of spirochetes. Wild-type mice vaccinated with BBK32 had fewer Borrelia-positive joints and ears (but not bladder cultures) compared to adjuvant alone or infection only controls (FIG. 1A). Decorin-deficient mice immunized in the same manner were completely protected in all tissues examined with the exception of one ear culture (20% positive) (FIG. 1B). This is markedly different from decorin-deficient control mice infected with Borrelia (p<0.0476; Fisher's exact test) for all tissues examined.

These data suggest that a Lyme vaccine composed of BBK32/DbpA or components thereof may be a more effective vaccine treatment than single-component formulations.

### Example 3

Vaccination of Wild-Type Mice with BBK32/DbpA Combination

While BBK32 alone had the ability of increasing resistance to Borrelia in wild-type mice, the almost complete protection afforded by BBK32 vaccination in decorin-deficient mice suggested that an immune response directed against one adhesin coupled with the inability to bind to a second could dramatically affect disease outcome. This further suggested that a combination vaccine composed of DbpA and BBK32 may be a more efficient vaccine candidate than single component formulations because the focus of the immune response would be directed against two adhesins. Antibodies generated as a result of vaccination could confer protection as a result of antibody-dependant cell-mediated cytotoxicity or by preventing spirochete adhesion to the host ECM. A multiple component vaccine would also be more likely to protect against heterologous Borrelia strains since differences between the adhesin sequences among the Borrelia may more likely be reconciled with 2 or more MSCRAMM candidates.

This hypothesis was tested by vaccinating wild-type mice with a combination of BBK32/DbpA prior to Borrelia infection and determining the percentage of Borrelia-positive blood cultures in the vaccinated mice.

Wild-type mice were immunized with 20 μg of BBK32 or SdrF in complete Freund adjuvant or with adjuvant alone as described (Cassatt et al. 1998). Mice vaccinated with both BBK32 and DbpA received 10 μg of each protein. Two weeks after the second immunization, mice were infected i.d. with 10⁴ spirochetes at the base of the tail. One week later, blood was collected and cultured for the presence of spirochetes and 2 weeks later, the mice were sacrificed and the ear, bladder, and one joint were examined for the presence of spirochetes.

Results

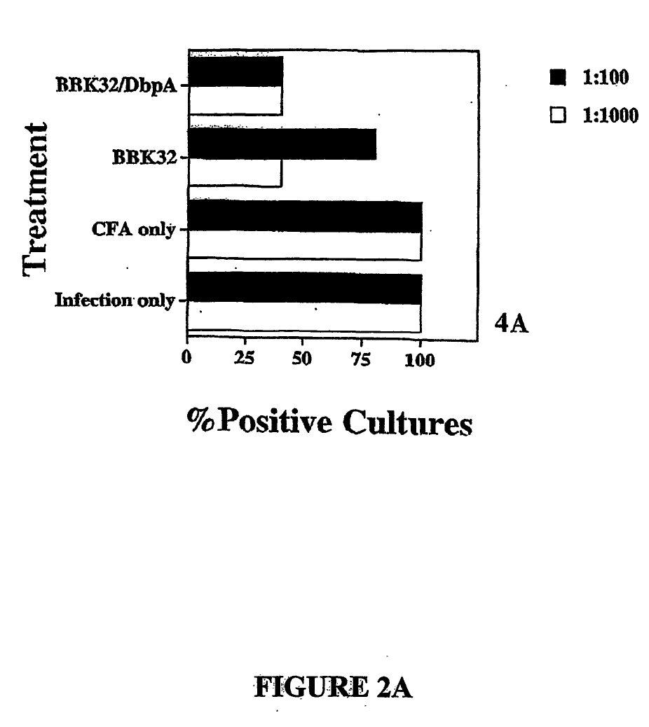

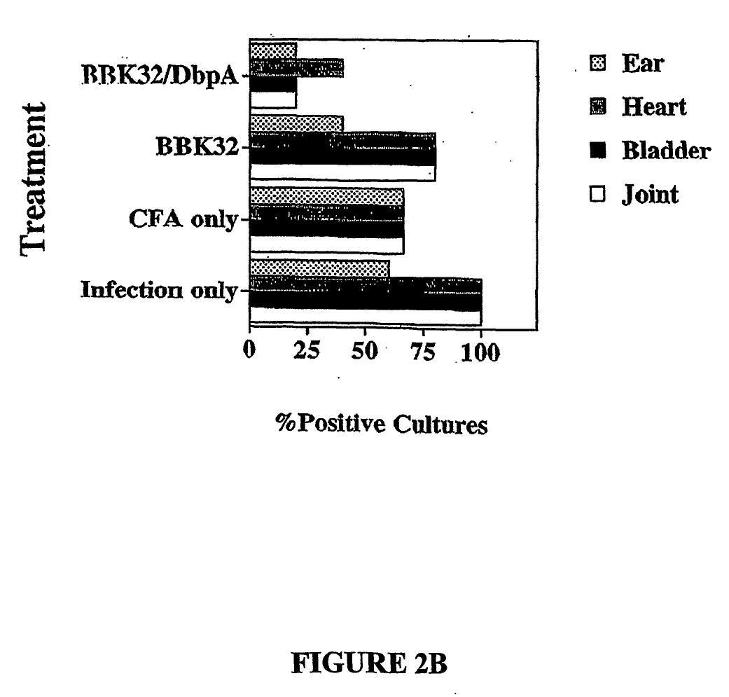

Blood collected from BBK32- and DbpA/BBK32-vaccinated mice and cultured for the presence of B. burgdorferi had fewer positive blood cultures than untreated or adjuvant only-treated mice (FIG. 2A). The proportion of Borrelia-positive tissues, however, was only dramatically reduced in mice receiving the multi-component formulation compared to BBK32-vaccinated and control mice (FIG. 2B).

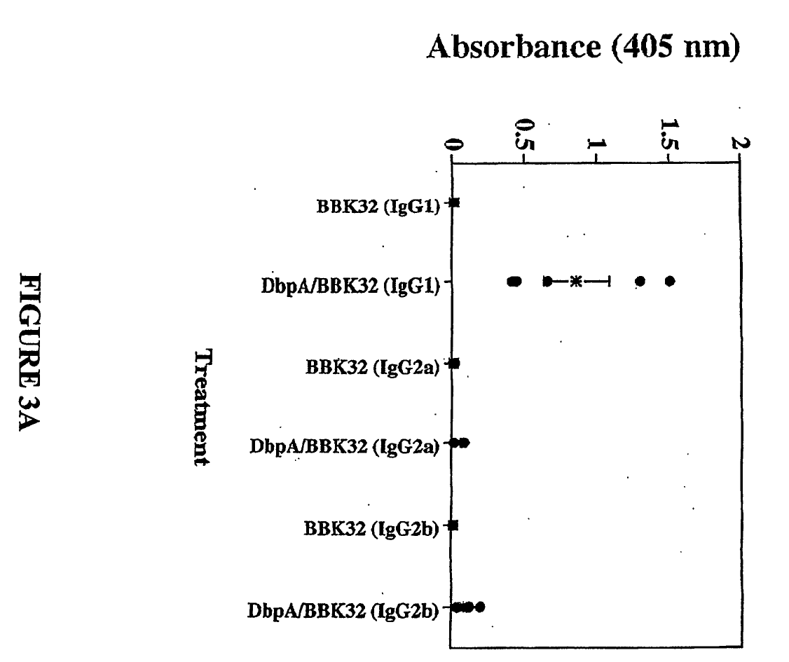

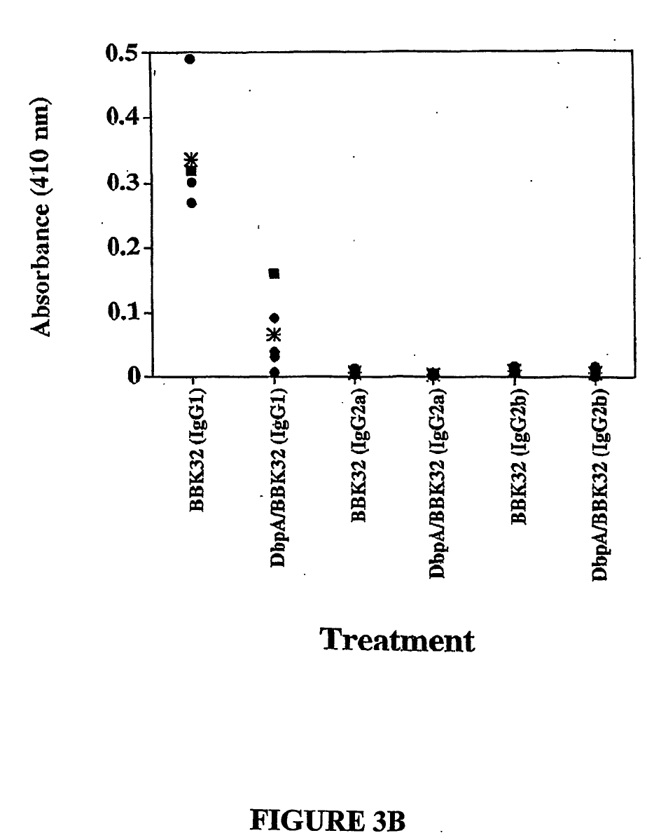

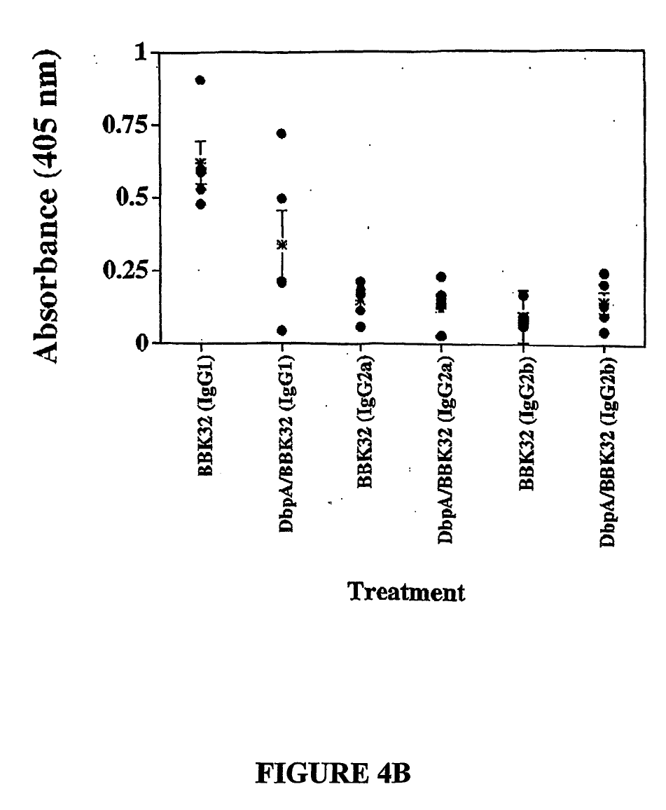

Mice vaccinated with BBK32 or with DbpA/BBK32 developed measurable antibody responses to both antigens (FIGS. 3A, 3B, 4A, and 4B). While both humoral and cellular mechanisms may be involved in protection against Borellia infections, these data do not distinguish which mechanism or combination thereof was responsible for conferring protection against infection. A more polarized cellular response to one antigen and a more polarized humoral response to the second may result in a more efficacious formulation compared to single component vaccines. Even though the same amounts of DbpA and BBK32 were used in the multi-component vaccinated mice, higher levels of IgG1 in particular were generated against DbpA than to BBk32. Furthermore, antibodies generated against more than one MSCRAMM could confer protection not only as a result of antibody-dependent cell-mediated cytotoxicity but also by preventing spirochete adhesion to the host ECM. A mltiple component vaccine would also be more likely to protect against heterologous Borrelia strains since differences between the adhesin sequences among the Borrelia may more likely be reconciled with two MSCRAMM candidates.

### Example 4

Further Advantage of a Combinatorial Vaccine

Laboratory studies have previously shown that decorin-deficient mice infected with Borrelia showed lower bacterial colonization of joints and lower incidence and severity of arthritis (Brown et al, 2001). In order to determine if the new combinatorial vaccine (DbpA/BBK32) is likely to be linked with any adverse effects such as increased arthritis severity, an arthritis assessment was conducted.

The joints collected from BBK32-, DbpA/BBK32-, or adjuvant only-vaccinated mice later is infected with 10⁴ B. burgdorferi (read above) were subjected to histopathological examination of formalin-fixed hind tibiotarsal joint samples. The tissues were embedded in paraffin and stained with hematoxylin and eosin. To determine the severity of arthritis, joints were scored according to the levels of neutrophil infiltrate as follows: 0, no arthritis, 1, minimal or rare (<10% tissue involvment), 2, mild (10-20%), 3, frequent (20-50%), and 4, severe (>50%).

TABLE II
Arthritis Development Following Infection with 10⁴ Borrelia
burgdorferi^{a} at 14 Days Post Infection.

| Vaccination | Mean arthritis rating | Arthritis Frequency (%) |
|---|---|---|
| BBK32/DbpA | 1.8^{c} | 3/5 (60%) |
| (0, 0, 0, 2, 2, )^{b} | | |
| BBK32 | 2.6 | 5/5 (100%) |
| (0, 0, 0, 0, 1)^{b} | | |
| Infection Only | 3.2 | 5/5 (100%) |
| (1, 1, 2, 3, 3)^{b} | | |
| CFA | 3.3 | 3/3 (100%) |
| (2, 3, 4)^{b} | | |

^{a}BALB/c mice were infected with 10⁴ B. burgdorferi strain 31 (passage 5) intradermally at the base of the tail.
^{b}Individual arthritis rating for each mouse.
^{c}Not significant.

Histological analysis of hind tibiotarsal joint also revealed difference in arthritis incidence and severity in multi-component vaccinated mice. These animals had a 60% arthritis incidence compared to 100% incidence in BBK32-vaccinated and control mice (Table II). Furthermore, the mean arthritis score was 1.8 in multi-component vaccinated mice compared to 2.6 in BBK32-vaccinated mice and >3.2 in the control groups (Table II).

In conclusion, the advantages of this invention/formulation over the existing Lyme vaccine are multiple. First, both BBK32 and DbpA are upregulated and expressed within the mammalian host. This provides the immune system with a target during the course of infection (as opposed to the duration of a blood-meal). Because these MSCRAMMs are expressed in the mammalian host, an MSCRAMM-derived vaccine has the potential of having therapeutic value even if administered after infection to date no correlation between immune responses and either DbpA or BBK32 have been linked with any adverse effects such as increased arthritis severity or autoimmune disease.

While the compositions and methods of this invention have been described in terms of preferred embodiments, it will be apparent to those of skill in the art that variations may be applied to the process described herein without departing from the concept, spirit and scope of the invention. All such similar substitutes and modifications apparent to those skilled in the art are deemed to be within the spirit, scope and concept of the invention as it is set out in the following claims.

### REFERENCES

The following references have been cited in the present application and are incorporated in their entirety by reference.

Barbour, A. G. “Isolation and cultivation of Lyme disease spirochetes.” Yale J. Biol. Med. 57:521-525, 1984.

de Silva, et al., “Borrelia Burgdorferi OspA is an Arthropod-Specific Transmission-Blocking Lyme Disease Vaccine,” J. Exp. Med., 183:271-275, 1996.

Genovese, et al., “Construction of DNA Sequences Complementary to Rat .alpha.sub.1 and .alpha.sub.2 Collagen mRNA and Their Use in Studying the Regulation of Type I Collagen Synthesis by 1,25-Dihydroxyvitamin D.sup.+,” Biochemistry, 23:6210-6216, 1984.

Jauris-Heipke, et al., “Genetic Heterogeneity of the Genes Coding for the Outer Surface Protein C (OspC) and the Flagellin of Borrelia Burgdorferi,” Med. Microbial. Immunol. (Berl), 182(1):37-50, 1993.

Oldberg, et al., “A Collagen-Binding 59-kd Protein (Fibromodulin) is Structurally Related to the Small Interstitial Proteoglycans PG-S1 and PG-S2 (Decorin),” The EMBO Journal, 6(9):2601-2604, 1989.

Schonherr, et al., “Interaction of Biglycan with Type I Collagen,” J. Biol. Chem., 270(6):2776-2783, 1995.

Schonherr et al., “Decorin-Type I Collagen Interaction,” J. Biol. Chem., 270(15):8877-8883, 1995.

Takeuchi, et al., “Bone Matrix Decorin Binds Transforming Growth Factor-.beta and Enhances its Bioactivity,” J. Biol. Chem., 269:32634-32638, 1994.

Guo, B., Hook, M., Norris, S. J. and Howell, J. “Borrelia burgdorferi: Adherence of Two Outer Surface Proteins to the Proteoglycan, Decorin”, Meeting of the Texas Branch of The American Society for Microbiology, Austin, Tex., Abstract No. 15, Nov. 11-13, 1993.

Alon, R., E. A. Bayer and M. Wilchek, “Biotin-Containing Protein as a Cause of False Positive Clones in Gene Probing with Streptavidin/Biotin”, BioTechniques, 14(2):209-210, 1993.

Isberg, Ralph R., “Discrimination Between intracellular Uptake and Surface Adhesion of Bacterial Pathogens”, Science, 252:934-938, May 17, 1991. “Immunological Screening of Expression Libraries”, Screening Expression Libraries with Antibodies and Oligonucleotides, pp 12.16-12.14, 1989.

Kantor, Fred S., “Disarming Lyme Disease”, Scientific American, pp. 34-39, Sep., 1994.

Kreis, T., Vale, R., “Guidebook to the Extracellular Matrix and Adhesion Proteins”, Decorin (DCN), pp. 48-49 Oct. 7, 1993.

Langermann, S., Palaszynski, S., Sadziene, A., Stover, .K, Koenig, S., “Systemic and Mucosal Immunity Induced by BCG Vector Expressing Outer-Surface Protein A of Borrelia burgdorferi”, Nature, 372:552-555, Dec. 8, 1994.

Philipp, M., Johnson B., “Animal Models of Lyme Disease: Pathogensis and Immunoprophylaxis”, Trends in Microbiology, 2(11):431-437, Nov., 1994.

Sharon, N., Lis, H., “Carbohydrates in Cell Recognition”, Scientific American, pp. 82-89, Jan., 1993.

Steere, A., “Lyme Disease: A Growing Threat to Urban Populations”, Proc. Natl. Acad. Sci., USA, 91:2378-2382, Mar., 1994.

Guo, B., Norris, S. J., Howell, J., Hook, M., “Identification of Decorin Binding Proteins on the Outer Membrane Surface of Borrelia burgdorferi,” Abstr. Annu. Meet. Am. Soc. Microbiol., D-161, p. 124, May, 1994 (listed in C10).

Guo, B., Norris, S. J., Rosenberg, Lawrence C., Hook, M., “Adherence of Borrelia burgdorferi to the Proteoglycan Decorin”, Infect. Immun., 63(9):3467-3472 (listed in C10), 1995.

Bidanset et al., “Binding of the Proteoglycan Decorin to Collagen Type VI,” J. Biol. Chem. 267(8):5250-5256, Mar. 15, 1992.

Guo et al., “Evidence that the decorin binding protein of Borrelia burgdorferi is an adhesin,” 96th General Meeting of the American Society for Microbiology, New Orleans, La., May 19-23, 1996, 248. ISSN: 1060-2011, XP 000618881, Abstract No. D-38.

Krumdieck et al., “The proteoglycan decorin binds Clq and inhibits the activity of the C1 complex.sup.1”, J. Immunol., 149(11):3695-3701, 1992.

Probert et al., “Immunization with the outer surface proteins of Borrelia burgdorferi provides limited cross-protection,” 95th ASM General Meeting, 144 ISSN 0067-2777, Abstract No. E-56, p. 290, May 21-25, 1995.

Yamaguchi et al., “Negative regulation of transforming growth factor-.beta. by the proteoglycan decorin,” Nature, 346:281-284, Jul. 19, 1990.

O'Brien et al. “HIV-1 tropism for mononuclear phagocytes can be determined by regions of gp120 outside the CD4-binding domain”, Nature, vol. 348(1), p. 69-73, 1990.

Stuber et al. “Assessment of major histocompatibility complex class I interaction with Epstein-Barr virus and human immunodeficiency virus peptides by elevation of membrane H-2 and HLA in peptide loading-deficient cells”, Eur. J. Immunol. vol. 22, p. 2697-, 1992.

Yaron et al. “Synthesis and immunological properties of the oligolysly-N′-dinitrophenyllysine and oligolysylalanylalanyl-N′-dinitrophenyllysine peptide series”, Biochemistry, vol. 13(2), p. 347-354, 1974.

Mayer et al. “Block oligopeptides (L-Lysyl)m-(L-Alanyl)n-L-Tyrosyl-(L-Alanyl)n-(L-Lysyl)m. II. circular dichroism and pulse-fluorimetry conformational studies”, Biopolymers, vol. 17, p. 337-360, 1990.

Brown, E. L., M. W. J. B. Johnson, A. Smith, K. G. Danielson, R. Iozzo, J. Piesman, B. P. Guo, J. J. Weis, and M. Hook. “Resistance to Lyme disease in decorin deficient mice.” J. Clin. Invest. In press 2001.

Cassatt, D. R., N. K. Patel, N. D. Utbrandt, and M. S. Hanson. “DbpA, but not OspA, is expressed by Borrelia burgdorferi felt during spirochetemia and is a target for protective antibodies.” Infect. Immun. 66:5379-5387, 1998.

Danielson, K. G., H. Baribault, D. F. Holmes, H. Grahm, K. E. Kadler, and R. V. Iozzo. “Targeted disruption of decorin leads to abnormal collagen fibril morphology and skin fragility.” J. Cell Biol. 136:729-743, 1997.

Guo, B., S. J. Nonis, L. C. Rosenberg, and M. Höök. “Adherence of Borrelia burgdorferi to the proteoglycan decorin.” Infect. Immun. 63:3467-3472, 1995.

Guo, B. P., E. L. Brown, D. W. Dorward, L.C. Rosenberg, and M. Höök. “Decorin binding adhesins from Borrelia burgdorferi” Mol. Microbiol 30:711-723, 1998.

Hanson, M. S., D. R. Cassatt, B. P. Guo, N. K Patel, M. P. McCarthy, D. W. Dorward, and M. Höök. “Active and passive immunity against Borrelia burgdorferi decorin binding protein A (DbpA) protects against infection.” Infect. Immun. 66:2143-2153, 1998

Maniatis, T., E. F. Fritsch, and J. Sambrook. “Molecular cloning: A laboratory manual.” Cold Spring Harbor Laboratory Press, Cold Spring Harbor, N.Y. 1989.

Patti, J. M., B. L. Allen, M. J. MeGavin, and M. Höök. “MSCRAM—mediated adherence of microoorganisms to host tissues.” Annu. Rev. Microbiol. 48:585-617, 1994.

Probert, W. S., and B. J. B. Johnson. “Identification of a 47 kDa fibronectin-binding protein expressed by Borrelia burgdorferi isolate B31.” Mol Micro. 30:1003-1015, 1998.

1. A composition comprising an isolated decorin binding protein or decorin binding peptide and an isolated fibronectin binding protein or fibronectin binding peptide.

2. The composition of claim 1, wherein said isolated decorin binding protein or decorin binding peptide comprises the amino acid sequence of SEQ ID NO:4 and said isolated fibronectin binding protein or fibronectin binding peptide comprises the amino acid sequence of SEQ IID NO:2.

3. The composition of claim 1, wherein said isolated decorin binding protein or decorin binding peptide comprises an amino acid sequence of at least 10 amino acids from SEQ ID NO:4 and said isolated fibronectin binding protein or fibronectin binding peptide comprises an amino acid sequence of at least about 10 amino acids from SEQ ID NO:2.

4. The composition of claim 3, further comprising a pharmaceutically acceptable carrier or diluent.

5. The composition of claim 3, further comprising one or more compounds selected from the group consisting of excipients and adjuvants.

6. An isolated fibronectin binding protein or fibronectin binding peptide comprising an amino acid sequence of at least about 10 amino acids residues from SEQ ID NO:2.

7. An isolated fibronectin binding protein or fibronectin binding peptide comprising an amino acid sequence of at least about 10 amino acids residues from SEQ ID NO:2, wherein said amino acid residues are located between residues 125 and 165 of SEQ ID NO:2.

8. A method to elicit an immunological response in an animal, said method comprising injecting an effective amount of a composition comprising an isolated fibronectin binding protein or fibronectin binding peptide and an isolated decorin binding protein or decorin binding peptide into the animal.

9. The method of claim 8, wherein said isolated fibronectin binding protein or fibronectin binding peptide comprises an amino acid sequence of at least about 10 amino acids from SEQ ID NO:2, and said isolated decorin binding protein or decorin binding peptide comprises an amino acid sequence of at least 10 amino acids from SEQ ID NO:4.

10. A method for preventing Lyme disease in a animal, comprising administering to the animal an effective amount of a composition, wherein said composition comprises an isolated decorin binding protein or decorin binding peptide and an isolated fibronectin binding protein or fibronectin binding peptide.

11. The method of claim 10, wherein said decorin binding protein is DbpA and fibronectin binding protein is BBK32.
The method of claim 10, wherein said vaccine comprises an isolated decorin binding protein or decorin binding peptide comprising an amino acid sequence of at least 10 amino acids from SEQ ID NO:4 and an isolated fibronectin binding protein or fibronectin binding peptide comprising an amino acid sequence of at least about 10 contiguous amino acids from SEQ ID NO:2.


SEQUENCE LISTING (4 sequences)

SEQ ID NO: 1 - DNA, 1065 bp, Borrelia burgdorferi
  CDS (1)..(1065): BBK 32
atgaaaaaagttaaaagtaaatatttggctttgggattactttttggttttataagttgtgatttattcataagatatgaaatgaaagaggaatcccctggcttatttgataagggaaactctattttagagactagcgaggaatctattaaaaagcctatgaataagaaaggtaaaggtaagattgctagaaagaaaggcaaaagcaaggtttctagaaaagaaccgtatattcatagtttaaaaagggactctgctaataaaagcaattttttacaaaaaaatgtaattttagaggaagaaagtttaaaaactgaattattaaaagagcaatctgagactagaaaagaaaaaatacaaaaacaacaagatgaatataaagggatgactcaaggaagtttaaattcccttagcggtgaaagtggtgaattggaggagcctattgaaagtaatgaaattgatcttactatagattctgatttaaggccaaagagttccttacaaggcattgcaggatcaaactctatttcatacactgatgaaatagaggaagaggattatgatcagtattatttagatgaatatgatgaagaggatgaagaggaaataagattaagcaatcgatatcaatcttatctagaaggtgttaaatataatgtagattcagcaattcaaacaattactaagatatataatacttatacattattttcaacaaagctaacccaaatgtattctacacgccttgacaactttgctaaagccaaagctaaagaagaagctgcaaagtttacaaaagaagaccttgaaaaaaatttcaagaccttattaaattatattcaagtaagtgtaaagactgcagcaaattttgtatacataaatgacacgcatgcaaaaaggaaattagagaacattgaagcagaaataaaaactttaattgcaaagatcaaagaacaatctaatttatacgaagcatataaagcaatagtaacgtcaatcttattaatgagggattctcttaaagaagtgcaaggtatcattgacaagaatggcgtttggtactaa

SEQ ID NO: 2 - protein, 354 aa, Borrelia burgdorferi
MKKVKSKYLALGLLFGFISCDLFIRYEMKEESPGLFDKGNSILETSEESIKKPMNKKGKGKIARKKGKSKVSRKEPYIHSLKRDSANKSNFLQKNVILEEESLKTELLKEQSETRKEKIQKQQDEYKGMTQGSLNSLSGESGELEEPIESNEIDLTIDSDLRPKSSLQGIAGSNSISYTDEIEEEDYDQYYLDEYDEEDEEEIRLSNRYQSYLEGVKYNVDSAIQTITKIYNTYTLFSTKLTQMYSTRLDNFAKAKAKEEAAKFTKEDLEKNFKTLLNYIQVSVKTAANFVYINDTHAKRKLENIEAEIKTLIAKIKEQSNLYEAYKAIVTSILLMRDSLKEVQGIIDKNGVWY

SEQ ID NO: 3 - DNA, 564 bp, Borrelia burgdorferi
  CDS (1)..(564): DbpA
atgattaaatgtaataataaaacttttaacaatttacttaaactaactatacttgttaacctacttatatcatgtggactaacaggagcaacaaaaatcaaattagaatcatcagctaaagccattgtagatgaaatagatgcaattaaaaaaaaggctgcttctatgggtgtaaattttgatgcctttaaagataaaaaaacgggtagtggggtatcagaaaatccattcatacttgaagcaaaagtgcgagctactacagtagcggaaaaattcgtaatagcaatagaagaggaagctactaaacttaaagaaactggaagtagtggtgaattctcagcaatgtatgatttaatgtttgaagtctcaaaaccattacaagaattgggaatacaagagatgacaaaaacagtctcaatggcagctgaagagaatcctccaactacagctcaaggagtgcttgaaattgcaaaaaaaatgagagaaaaattacaaagggttcacaagaaaaaccaagacaccttaaagaaaaaaaataccgaagacagcactgctaaatcgtaa

SEQ ID NO: 4 - protein, 187 aa, Borrelia burgdorferi
MIKCNNKTFNNLLKLTILVNLLISCGLTGATKIKLESSAKAIVDEIDAIKKKAASMGVNFDAFKDKKTGSGVSENPFILEAKVRATTVAEKFVIAIEEEATKLKETGSSGEFSAMYDLMFEVSKPLQELGIQEMTKTVSMAAEENPPTTAQGVLEIAKKMREKLQRVHKKNQDTLKKKNTEDSTAKS